(19) United States
(12) Patent Application Publication
(10) Pub. No.: US 2025/0261971 A1
(43) Pub. Date: Aug. 21, 2025

(54) SPINAL TENSIONING SYSTEM, DEVICE, AND METHOD

(71) Applicant: Highridge Medical, LLC., Westminster, CO (US)

(72) Inventors: Randall G. Mast, Westminster, CO (US); Allison Christine Capote, Westminster, CO (US); John Spohn, Westminster, CO (US); Curtis Tade, Westminster, CO (US); Guillaume Quetier, Westminster, CO (US); Michael Vitale, Westminster, CO (US); Baron Lonner, Westminster, CO (US)

(21) Appl. No.: 19/059,813

(22) Filed: Feb. 21, 2025

Related U.S. Application Data
(60) Provisional application No. 63/556,278, filed on Feb. 21, 2024.

(51) Int. Cl. A61B 17/70

(52) U.S. Cl. CPC A61B 17/7022; A61B 17/7044

(57) ABSTRACT

A bone anchor and system for applying tension via a tether or cord between bone anchors is provided. An illustrative bone anchor includes a screw having a head and a shank, where the shank defines a screw axis. The bone anchor further includes a connector attachable to the head of the screw, where the connector is configured to receive and secure a cord to the bone anchor.

### CROSS-REFERENCE TO RELATED APPLICATION

The present application claims the benefit of and priority, under 35 U.S.C. § 119, to U.S. Provisional Application Ser. No. 63/556,278 filed Feb. 21, 2024, entitled “SPINAL TENSIONING SYSTEM, DEVICE, AND METHOD” the entire disclosure of which is hereby incorporated herein by reference, in its entirety, for all that it teaches and for all purposes.

### BACKGROUND

The present disclosure relates to surgical techniques and devices for use in correcting spinal deformities, such as scoliosis. More specifically, this disclosure discusses various vertebral implants for use in securing a flexible elongated member (e.g., tether or cord) between vertebral bodies and techniques for correction of spinal deformities using the disclosed implants.

Dynamic stabilization techniques, such as vertebral body tethering, are used in spinal treatment procedures for juveniles to permit enhanced mobility of the spine while also providing sufficient counter loading of a spinal curvature to effect treatment through bone growth modulation, particularly during times of rapid growth. Such dynamic stabilization systems may include fixed, uniaxial, or polyaxial bone anchors installed in adjacent or nearby vertebrae of the spine, various cord clamping devices, and a flexible cord secured to the bone anchors that may be tensioned between the bone anchors.

Current techniques and implants suffer from various deficiencies or potential failure modes as well as difficulties in implantation among other things. The following disclosure discusses various implants and surgical procedures to address these and other short comings with traditional approaches to spinal tethering.

### BRIEF SUMMARY

Embodiments of the present disclosure were contemplated to address the above-noted issues of existing stabilization techniques. This disclosure recognizes, among other things, that improving various aspects of spinal tethering systems can involve improvements such as implant (e.g., bone screw and/or anchor) revisions to mitigate screw migration, cord securing mechanisms (e.g., cord clamping) to reduce stress on the cord or reduce implantation time, construct strengthening to reduce or eliminate construct failure, and construct changes to prevent overcorrection, among others. Details of various concepts are provided below.

One aspect of the present disclosure provides a bone anchor, which may include a screw comprising a head and a shank, where the shank defines a screw axis, and a connector attachable to the head of the screw, where the connector is configured to receive and secure a cord offset from the screw axis.

In some embodiments, the connector can comprise a connector body and connector cap that moves relative to the connector body.

In some embodiments, the bone anchor can include a set screw that threads into the connector body to secure the connector cap to the connector body.

In some embodiments, the connector body can comprise a cord receiver and wherein a size of the cord receiver changes as the connector cap moves relative to the connector body.

In some embodiments, the connector body can comprise a cord receiver having an open top end to receive the cord.

In some embodiments, the connector cap can comprise a clamp that retains the cord between the connector body and the connector cap and that limits the cord from sliding through the clamp when the connector cap is secured into the connector body.

In some embodiments, the bone anchor can include a staple through which the screw is inserted.

In some embodiments, the staple can comprise an extending surface having at least one additional opening for a second screw.

In some embodiments, the connector can align the cord with a screw axis of the second screw.

In some embodiments, the staple can comprise an extension having a cord support face to receive and secure at least one of a second cord and a second cord between a post and the cord support face.

In some embodiments, the staple can comprise an alignment feature.

In some embodiments, the alignment feature can be provided on a ring that is rotatable relative to a bottom portion of the staple.

In some embodiments, the bone anchor can include an anchor plate having a first opening and a second opening.

According to at least another embodiment, a bone anchor can be provided that includes: a first screw; a second screw; an anchor plate coupling the first screw with the second screw; and a cord receiver configured to couple a cord or second cord under tension with one or both of the first screw and second screw.

In some embodiments, the cord receiver can be integrally formed with the anchor plate.

In some embodiments, the cord receiver can be part of the first screw.

In some embodiments, the cord receiver can comprise a tulip head having internal threads to receive a set screw.

In some embodiments, the cord receiver and the anchor plate can comprise teeth that substantially prevent a relative rotation thereof when a face of the anchor plate contacts a face of the cord receiver.

In some embodiments, the first screw can extend through a first opening of the anchor plate and wherein the second screw extends through a second opening of the anchor plate.

In some embodiments, the first screw can draw the cord receiver into contact with the anchor plate and wherein the cord receiver abuts the anchor plate to substantially prevent the second screw from withdrawing out of the anchor plate.

According to at least another embodiment, a bone anchor can be provided that includes: a screw comprising a screw axis; and a cord receiver that moves relative to the screw axis along a track provided adjacent to a head of the screw.

In some embodiments, the track can comprise a curved track.

In some embodiments, the track can comprise a linear track.

In some embodiments, the cord receiver can comprise a slot that mates with the track.

In some embodiments, the bone anchor can further include: a first set screw that substantially fixes a position of the cord receiver relative to the screw; and a second set screw that substantially holds a cord within a tulip head of the cord receiver.

In some embodiments, both the first set screw and the second set screw can utilize a common threading in the cord receiver.

In some embodiments, the second set screw can thread through a center of the first set screw.

According to at least another embodiment, a system can be provided that includes: two or more bone anchors; and at least one of a second cord and a looped cord that couples the two or more bone anchors under a tensile force.

In some embodiments, the two or more bone anchors can be coupled with a cord and a second cord.

In some embodiments, the two or more bone anchors can comprise a first bone anchor, a second bone anchor, a third bone anchor, and a fourth bone anchor.

In some embodiments, a cord can apply a first tensile force between the first bone anchor and the second bone anchor and wherein the second cord can apply a second tensile force between the third bone anchor and the fourth bone anchor.

In some embodiments, the first and third bone anchors can be implanted in a first vertebra and wherein the second and fourth bone anchors can be implanted in a second vertebra.

In some embodiments, a first cord can apply a first tensile force between the first bone anchor and the second bone anchor and wherein a second cord can apply a second tensile force between the third bone anchor and the fourth bone anchor.

In some embodiments, at least one of the first cord and the second cord can comprise the looped cord.

In some embodiments, the at least one of a second cord and a looped cord can wrap around a notch in one of the two or more bone anchors.

In some embodiments, the notch can be provided around an axis of a first bone anchor in the two or more bone anchors.

According to at least another embodiment, a system can be provided that includes: a screw; a staple; and a looped cord that wraps around the screw and applies a tensile force to the screw.

In some embodiments, the system may further include a locking nut that engages the staple and secures the looped cord therebetween.

In some embodiments, the system may further include a ring that engages the staple, wherein the ring is positioned around the screw and directly contacts the looped cord.

In some embodiments, the ring may comprise a cord receiver to receive and at least partially retain the looped cord therein.

In some embodiments, the ring may be configured to rotate relative to the staple.

In some embodiments, the ring can comprise a friction fit to engage the staple.

In some embodiments, the ring can comprise a ring stop to engage the staple.

In some embodiments, the system may further include a second screw coupled with the screw via an anchor plate.

In some embodiments, the screw and the second screw can comprise a same type of screw.

In some embodiments, the screw and the second screw can comprise a different type of screw.

In some embodiments, the looped cord may directly contact the screw.

In some embodiments, the anchor plate and the staple can comprise features that substantially prohibit relative rotation thereof when the anchor plate is contacting the staple.

In some embodiments, the system may further include a locking nut that secures the looped cord around the screw.

According to at least another embodiment, a bone anchor can be provided that includes: a staple having at least one opening to receive a screw and at least one feature to receive a second cord or looped cord and to counteract a tensile force therein.

In some embodiments, the at least one feature can comprise a cord support face and a post that presses the second cord or looped cord into the cord support face.

In some embodiments, the cord support face can be provided on an extension of the staple.

In some embodiments, the staple can comprise one or more arms for extending into a bony structure.

In some embodiments, the staple can comprise a cord support face that presses the second cord or looped cord into the screw.

According to at least another embodiment, a system can be provided that includes: a screw; and a cord coupler that couples at least two cord portions together and that couples the at least two cord portions with the screw.

In some embodiments, the screw can comprise a tulip head to receive the cord coupler.

In some embodiments, the system may further include a set screw that secures the cord coupler into a head of the screw.

In some embodiments, the cord coupler can comprise a hole that exposes the at least two cord portions to the set screw.

In some embodiments, the at least two cord portions may belong to a common second cord.

In some embodiments, the at least two cord portions may belong to different cords.

In some embodiments, the system may further include a second screw, wherein the cord coupler extends between the screw and the second screw.

In some embodiments, a first set screw may connect the cord coupler to the screw and wherein the second set screw may connect the cord coupler to the second screw.

In some embodiments, the first set screw can secure a first cord portion to the cord coupler and wherein the second set screw can secure a second cord portion to the cord coupler.

According to at least another embodiment, a bone anchor can be provided that includes: a screw shank; and a head that receives at least one cord and at least one second cord.

In some embodiments, the head can comprise at least one lateral post to receive the at least one second cord.

In some embodiments, the head can comprise two lateral posts.

In some embodiments, the at least one lateral post can be integrally formed with the head.

According to at least another embodiment, a surgical system can be provided that includes: a surgical instrument having: a proximal end; a distal end; and an instrument body coupling the proximal end with the distal end, wherein the distal end comprises a passage to support manipulation of a second cord around at least one bone anchor.

In some embodiments, the distal end can comprise a first second cord passage, a second cord passage, and a cord passage, wherein the proximal end can comprise a first opening and a second opening, wherein the first opening is coupled with the cord passage via a primary passage lumen in the instrument body, and wherein the second opening can be coupled with both the first second cord passage and the second cord passage via at least one secondary passage lumen in the instrument body.

In some embodiments, the distal end can interface with a tensioning tower when the tensioning tower is coupled with a first bone anchor.

In some embodiments, the second cord can wrap around a second bone anchor when the distal end interfaces with the tensioning tower.

In some embodiments, the second cord can wrap around a screw attachment tower when the screw attachment tower is coupled with a second bone anchor.

In some embodiments, the screw attachment tower can comprise a second cord notch to substantially prohibit axial movement of the second cord along a body of the tower.

In some embodiments, the screw attachment tower can further comprise a cord notch to allow a cord to pass from the first bone anchor to the second bone anchor.

In some embodiments, the tensioning tower can comprise a deflectable member that moves under application of a tensile force against the deflectable member.

In some embodiments, the tensioning tower can comprise a force sensor that measures the tensile force.

In some embodiments, the measurement of the tensile force can be displayed to a surgeon via a user interface.

In some embodiments, the surgical system may further include a tensioning tip having a spring and housing that is configured to be deflected by tension applied to a cord by a cord tensioner.

In some embodiments, the surgical system may further include a pivot ladder that translates the tensile force to a motion of one or more linkages in the pivot ladder.

In some embodiments, the bone anchor can comprise a cone implant into which a screw with a cord receiver is threaded into a central threaded lumen of the cone implant.

In some embodiments, the bone anchor can comprise a cone implant into which a screw with a cord receiver is threaded, further comprising a laser cut at a distal, narrow tip of the cone implant that can allow the tip to splay when the screw with a cord receiver is threaded into a central threaded lumen of the cone implant.

In some embodiments, the bone anchor can comprise a cone implant into which a screw with a cord receiver is threaded, further comprising one or more U-shaped laser cuts along a side wall of the cone implant that can allow the U-shaped laser cut region to splay outwards from the cone side wall of the cone implant when the screw with a cord receiver is threaded into a central threaded lumen of the cone implant.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a block diagram illustrating details of the system shown in FIG. 1;

FIGS. 3A-3H illustrate a bone anchor according to at least some embodiments of the present disclosure;

FIG. 4 illustrates another example of a bone anchor according to at least some embodiments of the present disclosure;

FIGS. 5A-5F illustrate another example of a bone anchor according to at least some embodiments of the present disclosure;

FIGS. 6A-6D illustrate another example of a bone anchor according to at least some embodiments of the present disclosure;

FIG. 7 illustrates various examples of coupling bone anchors according to at least some embodiments of the present disclosure;

FIG. 8 illustrates an example of a second cord according to at least some embodiments of the present disclosure;

FIGS. 9A-9C illustrate another example of a bone anchor according to at least some embodiments of the present disclosure;

FIGS. 10A-10E illustrate another example of a bone anchor according to at least some embodiments of the present disclosure;

FIGS. 11A-11C illustrate another example of a bone anchor according to at least some embodiments of the present disclosure;

FIGS. 12A-12C illustrate another example of a bone anchor according to at least some embodiments of the present disclosure;

FIGS. 13A-13E illustrate another example of a bone anchor according to at least some embodiments of the present disclosure;

FIGS. 14A-14B illustrate another example of a bone anchor according to at least some embodiments of the present disclosure;

FIG. 15 illustrates another example of a bone anchor according to at least some embodiments of the present disclosure;

FIGS. 16A-16D illustrate examples of bone anchor coupling according to at least some embodiments of the present disclosure;

FIG. 17 illustrates an example of coupling bone anchors together according to at least some embodiments of the present disclosure;

FIGS. 18A-18B illustrate another example of a bone anchor and bone anchor coupling according to at least some embodiments of the present disclosure;

FIG. 19 illustrate another example of coupling bone anchors together according to at least some embodiments of the present disclosure;

FIGS. 20A-20B illustrate a cord coupling system according to at least some embodiments of the present disclosure;

FIGS. 21A-21B illustrate details of a snake tip used in a surgical instrument according to at least some embodiments of the present disclosure;

FIGS. 22A-22C illustrate components of a surgical instrument according to at least some embodiments of the present disclosure;

FIGS. 23A-23C illustrate a mechanism to measure tension according to at least some embodiments of the present disclosure;

FIGS. 24A-24B illustrate an assembled surgical instrument according to at least some embodiments of the present disclosure;

FIG. 25 illustrates details of a component of a surgical instrument according to at least some embodiments of the present disclosure;

FIGS. 26A-26D illustrate a force sensor useable in a surgical instrument according to at least some embodiments of the present disclosure;

FIGS. 27A-27B illustrate details of an alternative snake tip used in a surgical instrument according to at least some embodiments of the present disclosure;

FIGS. 28A-28D illustrate another example of a bone anchor according to at least some embodiments of the present disclosure;

FIG. 29 illustrates another example of a bone anchor according to at least some embodiments of the present disclosure;

FIGS. 30A-30D illustrate an example of a staple useable with a bone anchor according to at least some embodiments of the present disclosure;

FIGS. 31A-31D illustrate another example of a bone anchor and a system comprising a plurality of bone anchors according to at least some embodiments of the present disclosure;

FIG. 32 illustrates details of a surgical instrument used to couple and manipulate bone anchors according to at least some embodiments of the present disclosure;

FIGS. 33A-33C illustrate details of a surgical instrument used to couple and manipulate bone anchors according to at least some embodiments of the present disclosure;

FIGS. 34A-34C illustrate additional details of a surgical instrument from FIGS. 33A-33C and FIG. 32;

FIGS. 35A-35C illustrate additional details of the surgical instrument from FIGS. 34A-34C;

FIGS. 36A-36C illustrate additional details of the surgical instrument from FIGS. 34A-34C;

FIGS. 37A-37C illustrate another example of a bone anchor comprising various cone implants according to at least some embodiments of the present disclosure; and

FIGS. 38A-38E illustrate another example of a bone anchor comprising various cone implants according to at least some embodiments of the present disclosure.

### DETAILED DESCRIPTION

Embodiments will now be described more fully with reference to the accompanying drawings. The present disclosure provides details on concepts for improving various aspects of a spinal tethering system, such as a spinal tethering system detailed in U.S. Pat. No. 10,653,453 titled “DYNAMIC STABILIZATION SYSTEMS AND ASSOCIATED METHODS”, which is hereby incorporated by reference in its entirety. The improvements discussed also relate to aspects of the spinal tethering instruments discussed in U.S. Pat. No. 10,905,474 titled “SURGICAL CORD TENSIONING DEVICES, SYSTEMS, AND METHODS” and U.S. Pat. No. 10,939,941 titled “SURGICAL CORD TENSIONING DEVICES, SYSTEMS, AND METHODS”, which are both hereby incorporated by reference in their entirety. The improvements discussed also relate to aspects of the spinal tethering devices discussed in U.S. Patent Publication No. 2022/0226023 titled “SPINAL TETHERING DEVICES, SYSTEMS, AND METHODS”, which is hereby incorporated by reference in its entirety.

The following description discusses various implants used in a spinal tethering system, such as bone screws, bone anchors, staples, and the like. Most spinal tethering systems utilize bone screw designs from traditional spinal fixation systems targeting similar portions of the spine, such as fixed head pedicle screws. These traditional bone screws were originally designed to couple solid spinal rods between vertebral bodies, and can induce unwanted stress on a flexible cord or tether when used in a spinal tethering system. Spinal fixation systems often include fixed, uni-axial, and multi-axial bone screws, where the screw head (e.g., tulip or saddle) is fixed or allowed to move (uni-axial or multi-axial) in reference to the shaft of the bone screw. Bone screws from spinal fixation systems were typically designed to receive spinal fusion rods rather than flexible elongate members, such as a cord or tether. The improvements outlined below are discussed in reference to these commonly available bone screw designs. The anchors referenced below involve an implant that is designed to surround at least a portion of a bone screw and engage surrounding bone surface to distribute loads from the bone screw over a greater area of cortical bone. Again, improvements outlined below to anchors are discussed in reference to these anchor or staple designs.

Bone screw and anchor designs discussed below can provide numerous benefits over traditional pedicle screws for use in spinal tethering. The benefits can include providing a stronger construct, reducing screw plow (e.g., movement/migration of the screw in the vertebral body over time), enhancing cord clamping, reducing cord breakage, reducing surgical procedure steps (eliminate multiple steps for separate anchor and screw placement), and providing for multiple mechanisms for coupling bone anchors to one another (e.g., to increase strength and provide redundancy).

Other devices discussed below include cord clamping mechanisms that deviate from use of set screws and typical tulip head pedicle screws. The cord clamping mechanisms discussed below provide benefits such as reducing stress points on the flexible cord, increasing holding strength, and allowing for controlled distribution of tension across multiple implant levels, among others. Additional benefits of each different implant is discussed below in reference to the illustrations of the specific designs.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

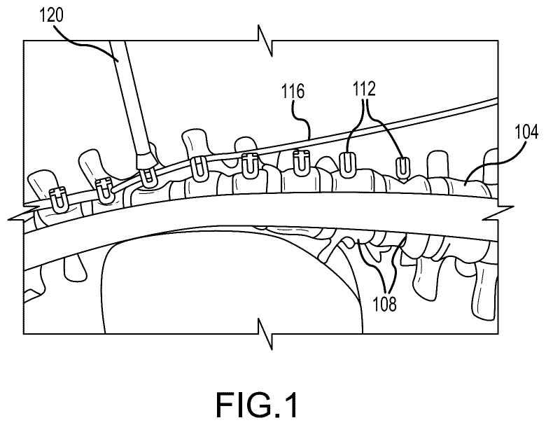

Turning initially to FIG. 1, a system is illustrated that may be used, for example, to correct a spinal deficiency of a patient. The system may include a plurality of bone anchors 112 implanted onto a patient anatomy. Illustratively, a bone anchor 112 may be implanted on one or more vertebra of the patient. Each bone anchor 112 may be implanted on a side of a vertebral body. The bone anchors 112 may be implanted on a lateral side of the vertebral body and, in some embodiments, may be implanted on the anterolateral aspect of the vertebral bodies on the convex side 104 of the curve formed in the patient's spine. Although the bone anchors 112 are illustrated as being implanted on the convex side 104 of the curve, it should be appreciated that one or more bone anchors 112 may be implanted on the concave side 108 of the curve formed in the patient's spine. For instance, a patient's spine may have multiple curves and it may be desirable to implant a plurality of cords 116 along different sides of the patient's spine. Bone anchors 112 may be implanted in the thoracic region and/or lumbar region. Illustratively, a bone anchor 112 may be implanted anywhere from T2-L5. The bone anchors 112 and the cord 116 may be implanted using one or more surgical instruments 120. The surgical instrument(s) 120 may support a minimally-invasive surgical procedure where multiple, but relatively small, incisions are made in the patient.

Although the term “cord” is used herein to refer to element 116, it should be appreciated that the term cord can encompass any element capable of applying tensile forces between two or more bone anchors 112. Thus, a cord 116 may include a cord, rope, tape, polymer line, or the like. The cord 116 may be capable of elastic deformation under tensile loads.

Moreover, the system including a plurality of bone anchors 112 and a cord 116 may be configured in a number of different suitable ways. In one example, each bone anchor 112 may fixedly secure a portion of the cord 116 such that the cord 116 is not released from a bone anchor 112 once secured.

In another example, one or more bone anchors 112 may be configured to flexibly secure a portion of the cord 116 such that if tensile forces applied to the cord 116 exceed a predetermined amount, then the cord 116 may be allowed to “slip” or move relative to the bone anchor 112. In such a configuration, the ability to allow the cord 116 to “slip” or move relative to the bone anchor 112 enables the entire implant system to share loads between bone anchors 112 rather than having more loads applied at certain bone anchors 112 than other bone anchors 112. In this manner, the likelihood of a cord 116 experiencing plastic deformation or total failure is reduced because the tensile forces are spread more evenly across the cord 116 instead of being concentrated at a particular location of the cord 116.

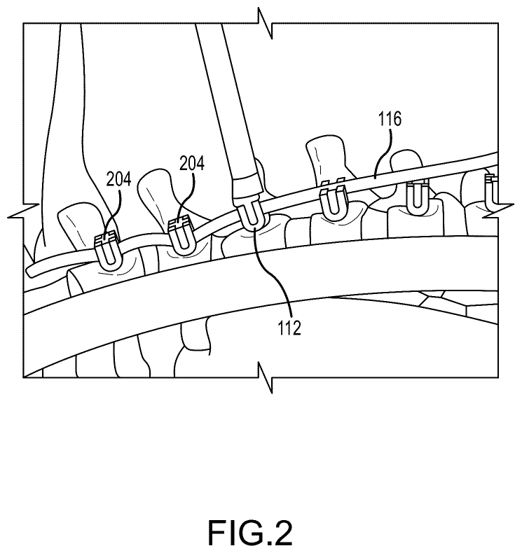

As shown in FIG. 2, the cord 116, which may also be referred to as a “tether”, can be received at each bone anchor 112 via a tulip head or similar type of cord receiver. The cord 116 may then be attached to the bone anchor 112 with a set screw 204. The set screw 204 may be placed over top of the cord 116. Tightening of the set screw 204 within the tulip head or cord receiver of the bone anchor 112 may enable a secure connection between the cord 116 and bone anchor 112. As tension is placed on the cord 116 then set screws 204 of adjacent bone anchors 112 are secured, a tension may be maintained in the cord 116 thereby causing tensile forces to be placed on adjacent bone anchors 112. The tensile forces, coupled with patient growth, may help to correct spinal curvature of the patient. As can be appreciated, these tensile forces may place additional stress on the bone anchor(s) 112 and/or the vertebral bodies in which the bone anchor(s) 112 are implanted. Various embodiments provided herein provide solutions to help prevent migration of the bone anchor(s) 112 through the bone and/or decoupling of the bone anchor(s) 112 from the cord 116.

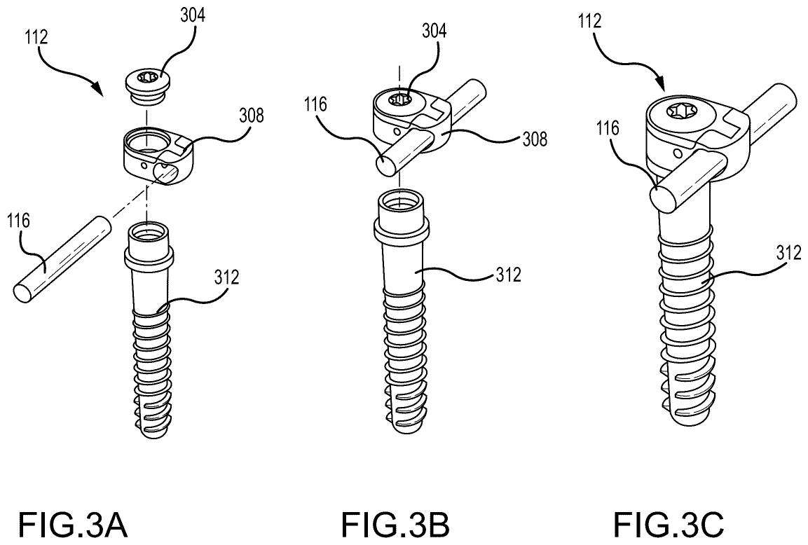

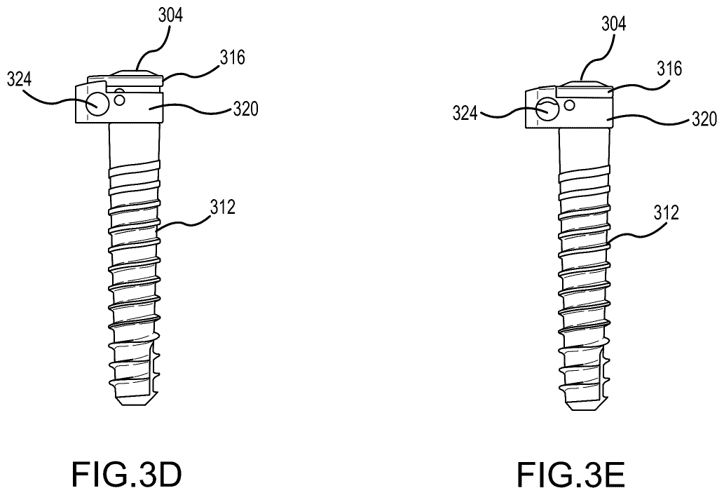

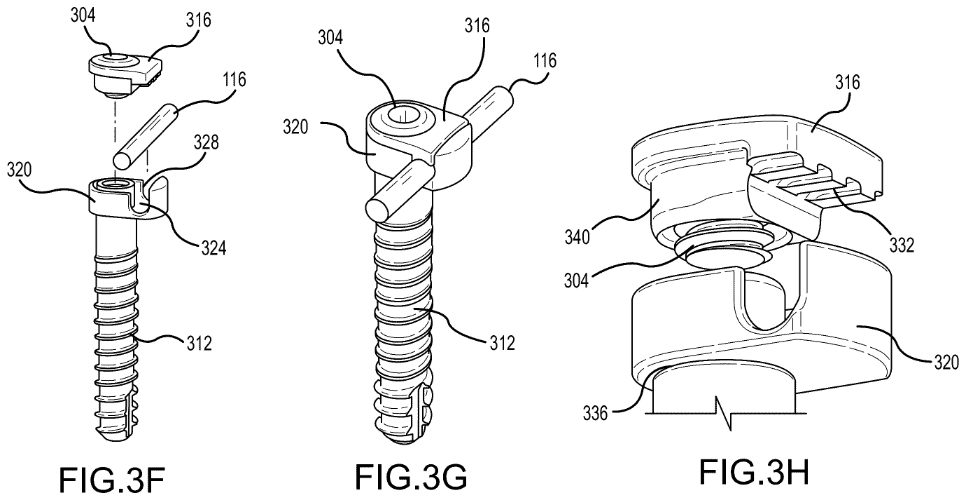

With reference now to FIGS. 3A-3H, a first example of a bone anchor 112 will be described in accordance with at least some embodiments of the present disclosure. The bone anchor 112 may be similar or have similar features to the other bone anchors depicted and described herein.

The bone anchor 112 illustrated in FIGS. 3A-3H exhibit a modular implant design that allows assembly of various implants customized to the patient and procedure. The bone anchor 112 is depicted to include a screw 312, a connector 308, and a set screw 304, The connector 308 may be constructed separately from the screw 312 and may be configured for attachment to the screw 312 even after the screw 312 has been implanted in a bone. In some embodiments, the connector 308 may comprise a modular design that enables different sizes or types of connectors 308 to be paired with different sizes or types of screws 312.

The set screw 304 may provide a couple of different functions for the bone anchor 112. In some embodiments, the set screw 304 comprises a threading that interfaces with threading on the screw 312. The set screw 304 may also hold the connector 308 onto a top of the screw 312 once the set screw 304 is threaded into the corresponding threading of the screw 312. The connector 308 may include a connector cap 316 and connector body 320 through which the set screw 304 passes to engage with the threading of the screw 312. While the threading of the screw 312 to receive the set screw 304 is shown to be internal threading and the threading of the set screw 304 is shown to be external threading, it should be appreciated that alternative configurations are possible where the threading of the screw 312 to receive the set screw is external threading and the threading of the set screw 304 is internal threading.

The connector 308 also includes a cord receiver 324 that presents a passage for the cord 116. The cord receiver 324 may be sized with an inner diameter that is larger than an outer diameter of the cord 116, thereby allowing the cord 116 to slide through the cord receiver 324. As shown in FIGS. 3D and 3E, as the set screw 304 is threaded into the screw 312 (e.g., further engaged and drawn into the body of the screw), the bottom of the set screw 304 may engage an upper surface of the connector cap 316. The connector cap 316 may be drawn downward (e.g., into the connector body 320 and toward the screw 312). Motion of the connector cap 316 as the set screw 304 is tightened may cause the connector cap to engage the cord 116 inside of the cord receiver 324. In other words, tightening of the set screw 304 may cause the connector cap 316 to clamp down onto the cord 116, thereby coupling the cord 116 with the bone anchor 112. When the set screw 304 is fully tightened into the screw 312, the diameter of the cord receiver 324 may be reduced and the cord 116 may be held between the connector cap 316 and the lower part of the cord receiver 324. The cord 116 may be pinched within the cord receiver 324 to prevent the cord 116 from moving relative to the bone anchor 112.

FIGS. 3F-3H illustrate an alternative configuration of a connector 308 whereby the connector 308 is capable of receiving the cord 116 via a top opening 328 in the cord receiver 324. This particular configuration of connector 308 still exhibits a connector body 320 and connector cap 316; however, the connector cap 316 in this particular configuration also comprises a clamp 332 to complete the cord receiver 324. The clamp 332 may also include a number of ribs or teeth that help engage the cord 116 when the cord 116 is positioned within the cord receiver 324. Tightening of the set screw 304 may cause the connector cap 316 to move downward, thereby closing the size of the cord receiver 324 and pinching the cord 116 within the cord receiver 324. Utilization of a connector 308 with a top opening 328 may facilitate easier access for the cord 116 into the cord receiver 324 since the cord 116 is no longer required to be threaded through the cord receiver 324. Instead, the screw 312 can be implanted and the connector body 320 can be attached to the top of the screw 312 before the cord 116 is placed into position in the cord receiver 324.

The connector body 320 may rest atop a body-screw interface 336 of the screw 312. In particular, the screw 312 may be provided with a shoulder or similar face for the body-screw interface 336 that supports the bottom surface of the connector body 320. Thus, tightening of the set screw 304 will not cause the connector body 320 to move relative to the screw 312. Instead, tightening of the set screw 304 will cause the connector cap 316 to move downward into the connector body 320 as the connector cap 316 also moves downward into the screw 312. The connector cap 316 may be retained within the connector body 320 with a cap fitting 340 that substantially prevents the connector cap 316 from rotating within the connector body 320. Thus, the connector cap 316 is only moving translationally (e.g., upward or downward) with respect to the connector body 320 while the set screw 304 is turned.

As tension is placed on the cord 116 (e.g., during installation) and a predetermined amount of tension is present in the cord 116, then the set screw 304 may be tightened to secure the cord 116 relative to the bone anchor 112. The connector 308 is configured to offset the cord receiver 324 from the axis of the screw 312. Offsetting the cord 116 from the axis of the screw 312 may help to relieve certain forces that might otherwise urge the screw 312 out of the vertebral body or cause the screw 312 to migrate through the vertebral body.

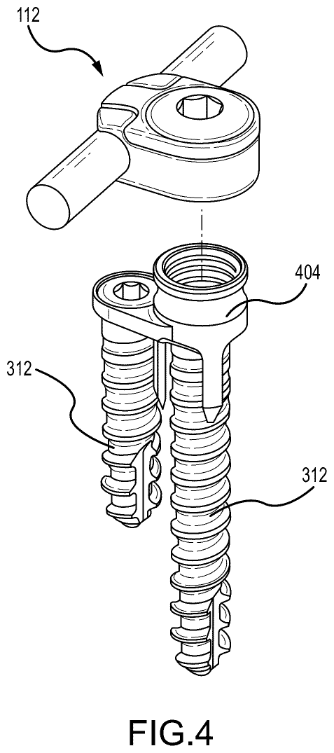

FIG. 4 illustrates another example of a modular bone anchor 112 according to at least some embodiments of the present disclosure. The bone anchor 112 is shown to include a plurality of screws 312 coupled together with an extending staple 404. In the illustrated configuration, the extending staple 404 includes a primary staple for interfacing with one of the screws 312 as well as an extension that interfaces with the other of the screws 312. In some embodiments, the portion of the extending staple 404 that includes the primary staple may also include a threaded receiver for connection with a connector 308. The illustrated configuration also shows that the connector 308, when attached to the threaded receiver of the extending staple 404, places the cord receiver substantially over top of the second screw 312. In this way, the connector 308 is provided with a connection strength of two screws 312 with a bony structure, thereby counteracting ploughing and other screw migration issues.

In some embodiments, the connector 308 may be placed over top of the extending staple 404, then the set screw 304 of the connector 308 may attach the connector 308 with the extending staple 404. Utilization of the extending staple 404 may also enhance the connection between the bone anchor 112 and the bony structure into which the anchor 112 is placed.

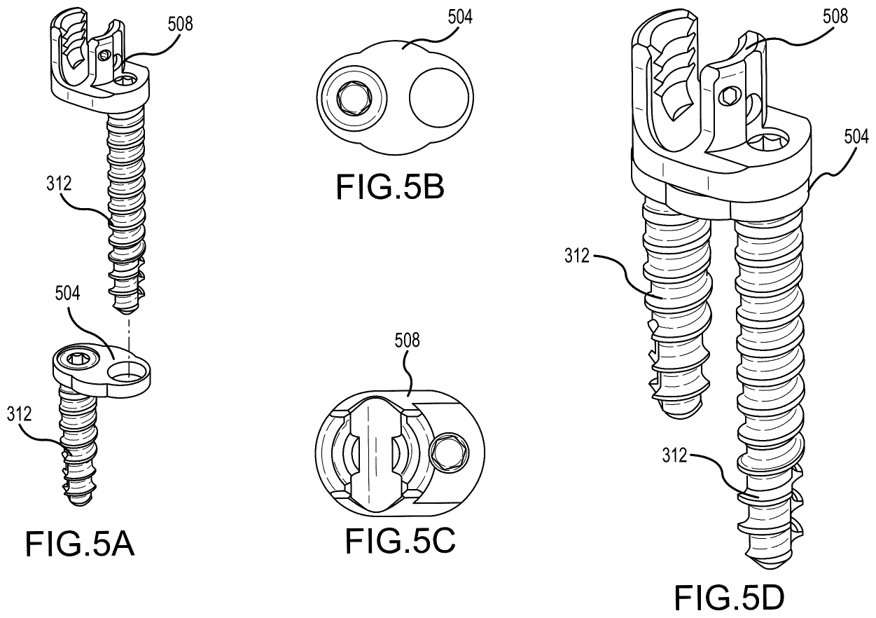

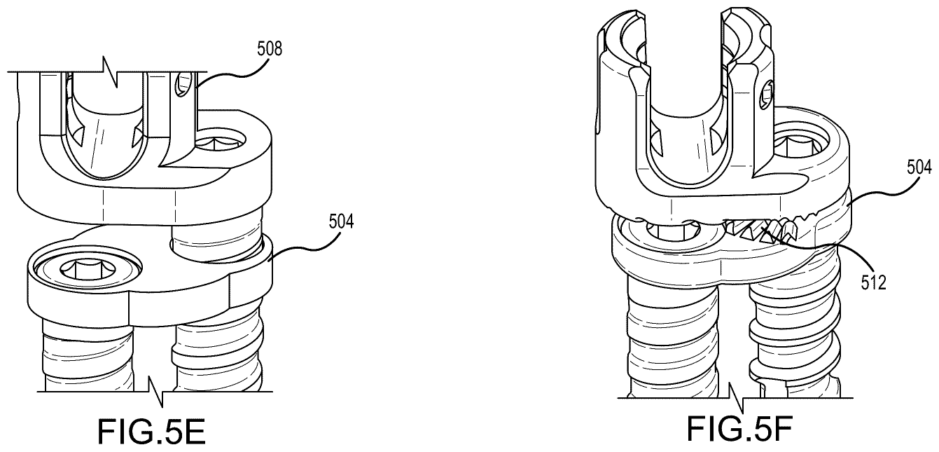

FIGS. 5A-5F illustrate still further possible configurations of a bone anchor 112 in accordance with at least some embodiments of the present disclosure. The bone anchor 112 illustrated herein is shown to include a first screw 312 connected with a cord receiver 508 and a second screw 312 connected with an anchor plate 504. FIG. 5D illustrates a configuration where the first screw 312 is placed through one hole of the anchor plate 504 to situate the cord receiver 508 on the anchor plate 504.

In some embodiments, the anchor plate 504 may include a piece of material having at least two holes, each of which is configured to receive a different screw 312. The cord receiver 508 may be similar to other cord receivers 324 or connectors 308 depicted and described herein. For instance, the cord receiver 508 may include a tulip head having an opening for receiving a cord 116. The tulip head of the cord receiver 508 may also include a threaded receiver to accept a set screw 304. As the set screw 304 is tightened into the threaded receiver of the cord receiver 508, the set screw 304 may substantially fix a position of the cord 116 relative to the cord receiver 508.

The first screw 312 that interfaces with the cord receiver 508 may be positioned within the cord receiver 508 such that axial motion of the screw 312 translates directly to axial motion of the cord receiver 508. The first screw 312 may be separate from the cord receiver 508 or may be integrally formed with the cord receiver 508.

The second screw 312 that interfaces with the anchor plate 504 may be positioned within the anchor plate 504 such that axial motion of the screw 312 translates directly to axial motion of the anchor plate 504. The second screw 312 may be separate from the anchor plate 504 or may be integrally formed with the anchor plate 504. Moreover, when the cord receiver 508 is positioned over the anchor plate 504, the second screw 312 may be substantially prohibited from being removed from the anchor plate 504. In other words, the cord receiver 508, when overlapping and abutting the anchor plate 504, may vertically fix the second screw 312 into the anchor plate 504.

FIGS. 5E and 5F illustrate two possible configurations of the anchor plate 504 and cord receiver 508. In one configuration, the anchor plate 504 may include a substantially planar top surface and the cord receiver 508 may include a substantially planar bottom surface for interfacing with and contacting the substantially planar top surface of the anchor plate 504. In another configuration, the anchor plate 504 and/or cord receiver 508 may be provided with interface features or locking features that help prohibit relative rotation between the anchor plate 504 and the cord receiver 508 when the two components abut one another. In particular, one or both of the anchor plate 504 and cord receiver 508 may be provided with teeth 512 that substantially help to secure a lateral connection between the anchor plate 504 and cord receiver 508.

In some embodiments, the anchor plate 504 includes a first set of teeth and the cord receiver 508 includes a second set of teeth that are designed to mate with (e.g., interlock, mesh, etc.) the first set of teeth provided on the anchor plate 504. Peaks of one set of teeth may be designed to mate with valleys of the other set of teeth when the anchor plate 504 abuts the cord receiver 508.

In some embodiments, the footprint of the anchor plate 504 and/or cord receiver 508 may be designed to fit within a vertebra. As an example, the footprint of the anchor plate 504 and/or cord receiver 508 may have a maximum width of 14.00 mm or less, thereby enabling the entire structure to be inserted through a Minimally Invasive Surgical (MIS) port or instrument.

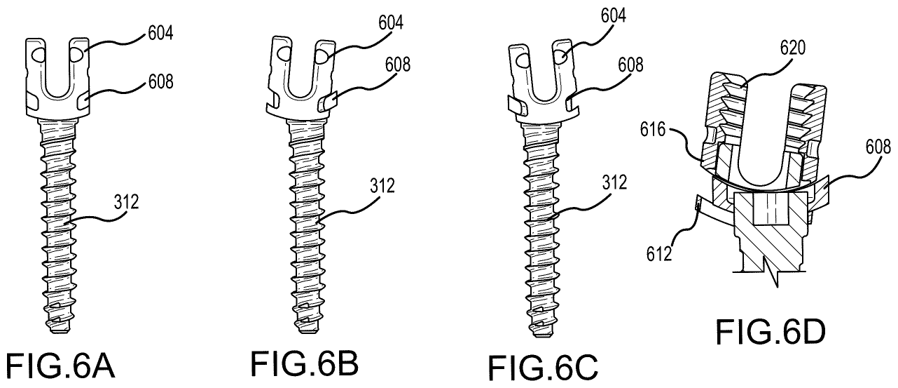

FIGS. 6A-6D illustrate another configuration of a bone anchor 112 in accordance with at least some embodiments of the present disclosure. The bone anchor 112 is shown to facilitate minor translations (e.g., lateral movements) of the cord 116. Specifically, and without limitation, the bone anchor 112 may include a screw 312, a cord receiver 604, and a track 608. The geometries of the cord receiver 604 and track 608 may create a cord housing that is capable of translating relative to the screw 312 shank to allow for ‘misalignment’ of screws while maintaining a straight cord 116 path. In this design, the cord receiver 604 moves on a curved surface (e.g., the track 608) within the medial lateral plane of the patient for a posterior approach to the spine, but it is the anteroposterior plane for the lateral approach described herein.

It may also be possible to design the track 608 with a flat configuration rather than an arcuate configuration. An advantage to using an arcuate track 608 is that translation of the cord 116 is enabled, but the instrument used to insert the screw 312 into bone can be inserted through the same port as the cord receiver 604. If not intended to be inserted separately then access to cord receiver 604 for instrument interface and set screw insertion can be achieved through the same port used to insert screw 312. It could also translate on a flat surface, but the benefit of the curve is that the set screw instrument could be inserted thru the same port.

During installation of the bone anchor 112, the screw 312 and cord receiver 604 may be driven together into the bony structure. Even with the screw 312 fully inserted into bone, the cord receiver 604 may be allowed to translate by moving along the track 608. As shown in FIG. 6D, the cord receiver 604 may have a feature that mates with the track 608 and slides along the track. In particular, the cord receiver 604 may have a slot with a top portion 616 positioned above the slot and a bottom portion 612 positioned below the slot. The dimensions of the slot separating the portions of the cord receiver 604 may be larger than the width of the track 608, thereby facilitating motion of the cord receiver 604 along the track 608.

The track 608 may be provided as a movable member of the cord receiver 604 and may have tabs that limit the amount of movement the track 608 can have relative to the cord receiver 604. The track 608 may include a hole therein that fits over top of the head of the screw 312. The track 608 may be assembled by sliding through slide slot and holding it in place over the head of the screw 312 by means of a compression saddle insert (not shown) that is inserted through the top cord receiver of the tulip head, or alternatively through a side slot of the tulip head and held in place by a shoulder or other engagement feature on the head of the screw 312. The interaction between the hole in the track 608 and the shoulder on the screw 312 head may hold the two components together.

The cord receiver 604, configured with a tulip head, may further include threading 620 to receive one or more set screws. For example, a first set screw may be used to push on the tulip head to secure the assembly (e.g., substantially fix a relative position of the cord receiver 604, track 608, and screw 312). A second set screw (either thru the center of the first set screw, or using the same housing threads 620) would lock the cord 116 within the tulip head of the cord receiver 604. Once the first and second set screws are tightened, the cord receiver 604 may stop translating relative to the screw 312 and the cord 116 may be substantially fixed within the cord receiver 604.

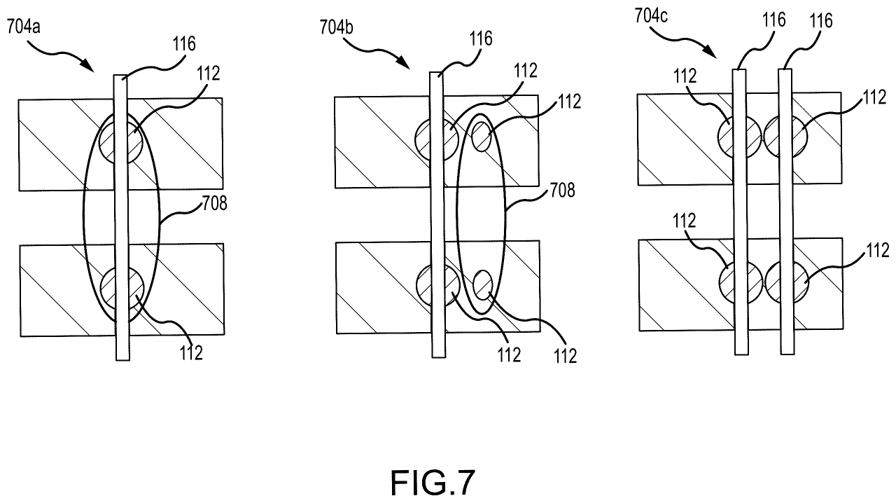

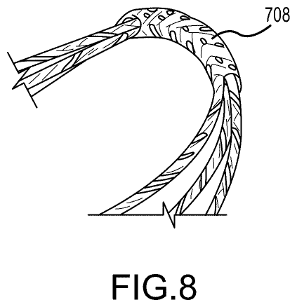

Referring now to FIGS. 7 and 8, additional details of other mechanisms for coupling two or more bone anchors 112 together will be described in accordance with at least some embodiments of the present disclosure. Up to now, a cord 116 has been described as the mechanism for coupling two or more bone anchors 112 via tensile forces. FIGS. 7 and 8 illustrate another coupling mechanism that can be used to apply tensile forces between two or more bone anchors 112.

Embodiments of the present disclosure contemplate use of a second cord 708 as a coupling mechanism. A second cord 708 may be used alone to couple two or more bone anchors 112 or a second cord 708 may be used in combination with another coupling mechanism (e.g., another second cord 708, a cord 116, combinations thereof, etc.).

A second cord 708 may be provided, in some embodiments, as a component to supplement a cord 116. A second cord 708 may be provided as a bundled second cord as shown in FIG. 8, or the second cord 708 may be constructed similarly to the cord 116. In some embodiments, a cord 116 may be used to couple all bone anchors 112 within a system, whereas a second cord 708 may be used as a supplement to the cord 116 to couple only particular bone anchors 112 in the system. Said another way, a second cord 708 may be provided to help increase tensile strength between bone anchors 112 at specific levels or segments of the patient's spine where increased forces are anticipated. The second cord 708 may be left in the system along with the cord 116. Alternatively, the second cord 708 may be used to aid in construction of the system (e.g., to provide a method of tensioning specific levels), but the second cord 708 may not be left in the patient after surgery. For instance, a second cord 708 may be used to hold correction of two adjacent bone anchors 112 during surgery, but may be removed during surgery as well.

A construct with supplemental fixation (e.g., a second cord 708 and a cord 116 coupled to common bone anchors 112) is shown as one example of fixation in FIG. 7. Other possible constructs of supplemental fixation are shown where two coupling mechanisms are connected to separate bone anchors 112 on the same vertebra. The two coupling mechanisms may include a cord 116 and second cord 708 or two cords 116.

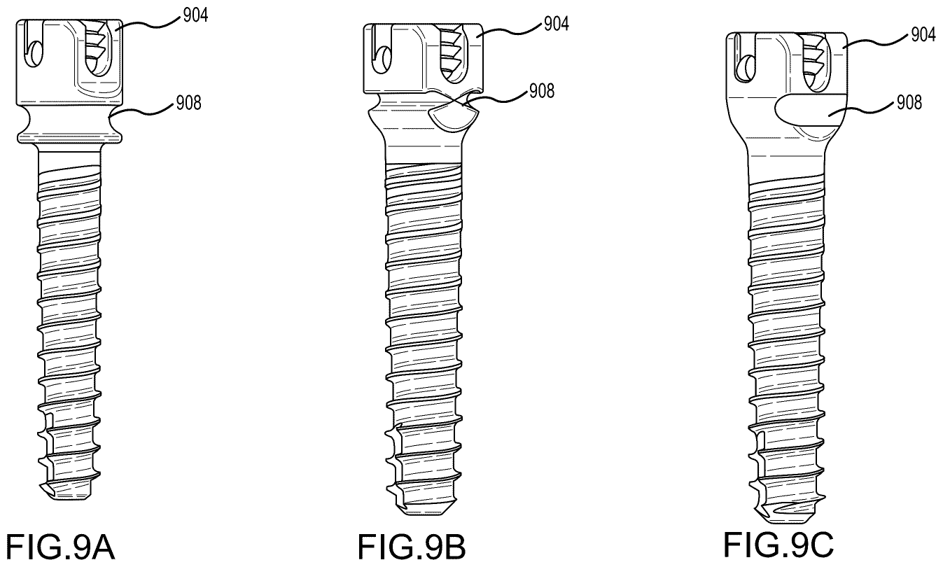

Because the present disclosure contemplates use of two different coupling mechanisms attaching to a single bone anchor 112, embodiments of the present disclosure also contemplate various examples of bone anchors 112 that accommodate multiple coupling mechanisms. A first example of a bone anchor 112 that accommodates two or more coupling mechanisms will be described in connection with FIGS. 9A-9C. In this particular configuration, the bone anchor 112 is shown to include a shank with a cord receiver 904 and notch 908. The notch 908 may be positioned between the shank and the cord receiver 904. In the illustrated embodiments, the notch 908 may wrap entirely around the circumference of the bone anchor 112 (see FIG. 9A). In another configuration, the notch 908 may wrap around the circumference of the bone anchor 112, but the cord receiver 904 may include one or more planar surfaces that interrupt a portion of the notch 908 (see FIG. 9B). In another configuration, two or more non-continuous notch features 908 may be provided at different sides of the cord receiver 904 (see FIG. 9C).

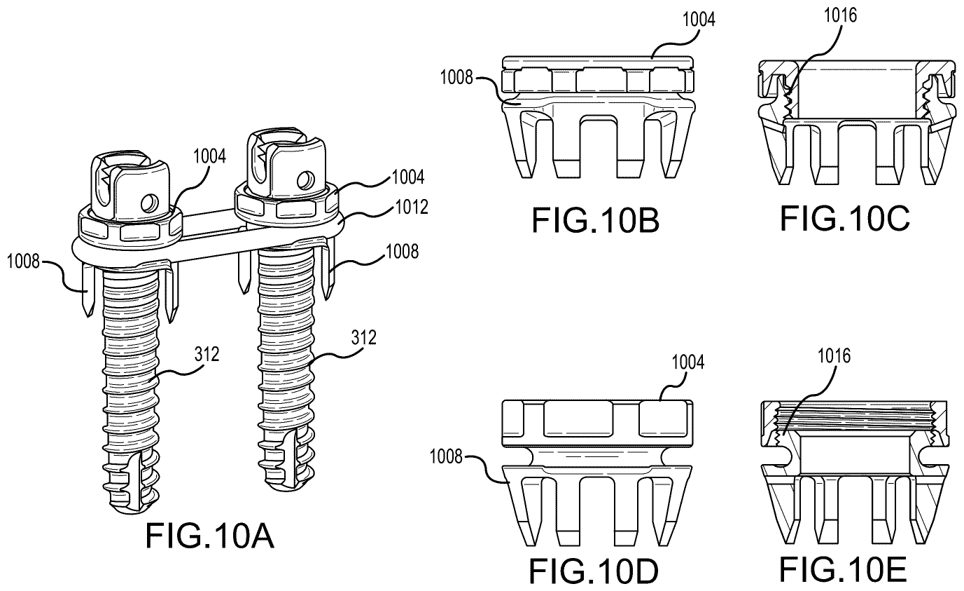

FIGS. 10A-10E illustrate an alternative configuration of bone anchors 112 that facilitate coupling by two or more coupling mechanisms. In particular, a bone anchor 112 is depicted with a modular configuration where a staple 1008 is provided with a receiver for a second cord 708, which may also be referred to as a looped cord 1012. The staple 1008 may comprise an annular ring to have a screw 312 pass therethrough. The staple 1008 may also include threads 1016 that interface with a locking nut 1004. The locking nut 1004 may also include threads 1016 to couple with threads 1016 of the staple 1008.

In some embodiments, the locking nut 1004, when screwed onto the staple 1008, may secure a looped cord 1012 therebetween. In particular, a looped cord 1012 may be used to couple two different screws 312 together and may be substantially secured to each screw 312 via the locking nut 1004 and staple 1008. FIG. 10A illustrates an example where each screw 312 is provided with its own locking nut 1004 and staple 1008, then the looped cord 1012 is configured to be secured between each the locking nut 1004 and staple 1008. The looped cord 1012 may be first secured to one of the screws 312, then tension may be placed on the looped cord 1012. After a predetermined amount of tension has been placed on the looped cord 1012, the locking nut 1004 around the other screw 312 may be tightened to secure the assembly of two screws 312 via the looped cord 1012.

Each screw 312 may be configured to pass through the staple 1008, meaning that an outer diameter of the screw 312 is less than an inner diameter of the staple. The screw 312 may interface with an inner surface of the staple 1008 (e.g., via a complimentary thread) or the inner surface of the staple 1008 may be substantially planar such that the staple 1008 freely passes over the shank of the screw 312 without interfacing with the threads 1016.

The locking nut 1004 may comprise an inner diameter that is less than a shoulder of the tulip head of the screw 312, thereby prohibiting the locking nut 1004 from passing above the tulip head of the screw 312. In some embodiments, the staple 1008 may be first implanted on the bony structure, then the locking nut 1004 may be placed over the shank of the screw 312. After the locking nut 1004 is placed over the shank of the screw 312, the screw 312 may be implanted in the bony structure through the staple 1008. A looped cord 1012 may be placed between the locking nut 1004 and staple 1008, then placed under tension before the locking nut 1004 is finally threaded down onto the staple 1008. Alternatively, or additionally, a different order of operations could be followed where screw 312 is not fully inserted/implanted into the bony structure (e.g., such that the tulip head of the screw 312 does not fully abut the locking nut 1004 in a locked position) until the looped cord 1012 is placed in its desired position. After placement of the looped cord 1012, the screw 312 may be fully inserted/implanted into the bony structure.

As shown in FIGS. 10B-10E, the threads 1016 may be realized in a number of different ways. In one example (e.g., FIGS. 10B and 10C), the locking nut 1004 may have threads 1016 on its outer diameter and the staple 1008 may have threads 1016 on its inner diameter. In another example (e.g., FIGS. 10D and 10D), the locking nut 1004 may have threads 1016 on its inner diameter and the staple 1008 may have threads 1016 on its outer diameter. Both the locking nut 1004 and staple 1008 may have platforms for pinching the looped cord 1012 therebetween.

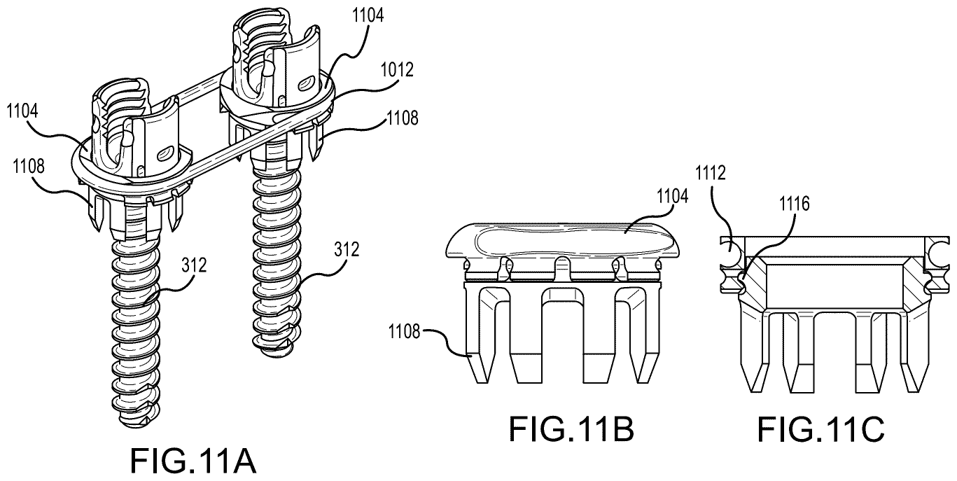

FIGS. 11A-11C illustrate another possible example of a staple 1108 that may be used to help limit an axial motion of a looped cord 1012 relative to a screw 312, in accordance with at least some embodiments. The embodiment of FIGS. 11A-11C exhibit a ring 1104 that retains the looped cord 1012. The ring 1104 may be assembled onto a top of the staple 1108 via a snap fit or friction fit 1116. The ring 1104 may be initially separate from the staple 1108 or may be assembled with the staple 1108. When the ring 1104 is snapped into place of the staple 1108, the ring 1104 may still be allowed to rotate about the staple 1108, but axial movement of the staple 1108 and ring 1104 may be substantially minimized or prohibited.

The configuration using a snap fit or friction fit 1116 enables the ring 1104 to be pressed into a mating configuration with the staple 1108 via the screw 312. In other words, as the screw 312 is inserted through the staple 1108, the shoulder of the tulip head of the screw 312 may drive the ring 1104 down onto the staple 1108. After the screw 312 has been driven into the staple 1108 enough, the ring 1104 may snap into place via the friction fit 1116. This snapping may secure the assembly of the screw 312, ring 1104, and staple 1108 from further axial movement. The ring 1104 may still rotate about the staple 1108 as the looped cord 1012 is placed around a cord receiver 1112 of the ring 1104.

The cord receiver 1112 may correspond to a notch or opening that is sufficiently sized to receive the looped cord 1012. The cord receiver 1112 may also include a geometry that snaps around the looped cord 1012. For example, the cord receiver 1112 may include a notch with lips that secure the looped cord 1012 within the notch of the cord receiver 1112. The notch and lips may prohibit the looped cord 1012 from leaving the cord receiver 1112 unless a substantial amount of force is placed thereon. Otherwise, the looped cord 1012 may slide around the ring 1104 in a relatively free range of motion, while still maintaining some contact with the ring 1104. In this particular configuration, the looped cord 1012 may be inserted after screw 312 insertion, but prior to an additional cord 116 being inserted and fixed between the tulip heads of the screw 312.

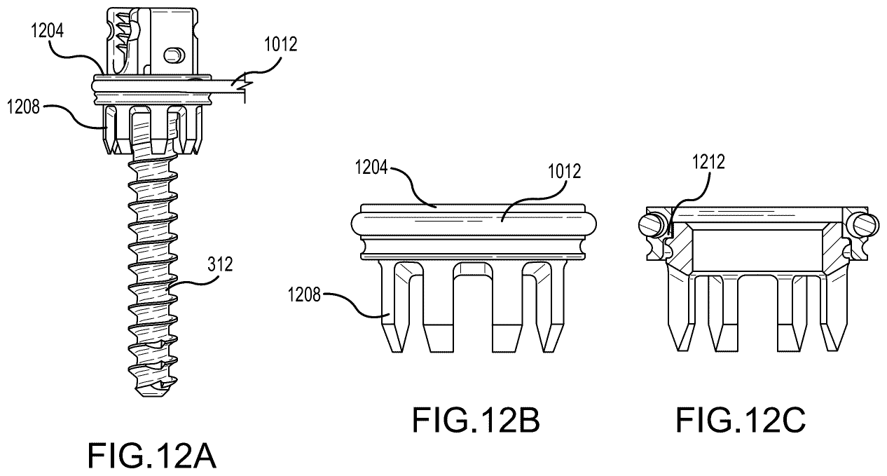

Referring now to FIGS. 12A-12C, yet another possible anchor configuration will be described in accordance with at least some embodiments of the present disclosure. The configuration of FIGS. 12A-12C is similar to the configuration of the bone anchor in FIGS. 11A-11C, except that the ring 1204 does not include a snap fit or friction fit with the staple 1208. The bone anchor is shown to include a ring 1204 having a cord receiver and a staple 1208 configured to receive the ring 1204. The ring 1204 and staple 1208 may include a ring stop feature 1212 that substantially prohibits the ring 1204 from passing beyond the staple 1208. The ring stop feature 1212 is different from a snap fit or friction fit 1116 in that the ring stop feature 1212 limits relative motion of the ring 1204 and staple 1208 in only one axial direction. Even when resting against the staple 1208, the ring 1204 may still be removed from the staple 1208 in an upward direction, unless a shoulder of the tulip head of screw 312 exists and screw 312 is inserted through anchor 1208.

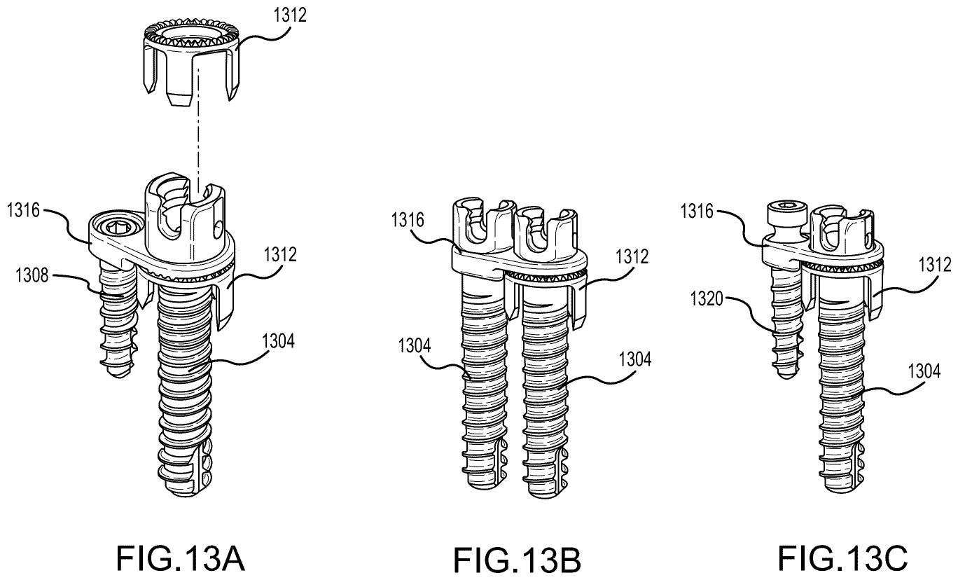

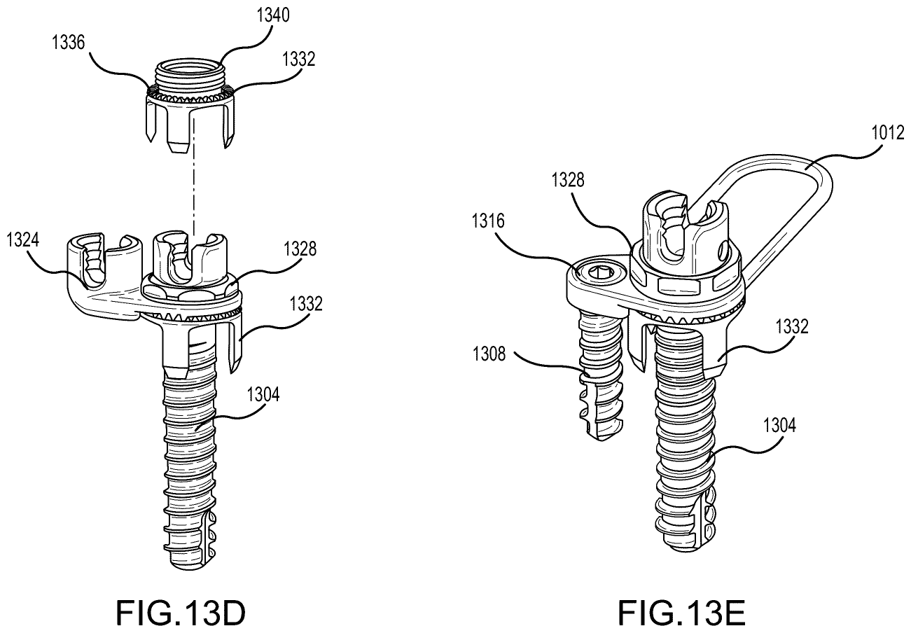

FIGS. 13A-13E illustrate further possible configurations of a bone anchor in accordance with at least some embodiments of the present disclosure. In particular, FIG. 13A is shown to include a first screw type 1304 and second screw type 1308 that are coupled to one another via a staple 1312 and anchor plate 1316 assembly. The first screw type 1304 is shown to include a tulip head for receiving a cord 116 whereas the second screw type 1308 is shown to include a flat top and does not directly interface with a cord 116. The second screw type 1308 may be provided to improve connectivity between the bone anchor and the bony structure and to limit migration of the bone anchor through the bony structure (e.g., to prevent ploughing).

The staple 1312 may be configured to have one of the screws (e.g., the first screw type 1304) pass therethrough. The staple 1312 may further include one or more features on its top surface that interface with mating features of the anchor plate 1316. The features on the top surface of the staple 1312 and the bottom surface of the anchor plate 1316 may substantially prohibit rotation of the two elements, when the features mate with one another.

The anchor plate 1316 may include two holes or openings. The first hole or opening may be configured to align with the opening of the staple 1312. The second hole or opening of the anchor plate 1316 may be configured to receive the second screw type 1308 or 1320. In the configuration of FIG. 13A, the head of the second screw type 1308 may sit below a top surface of the anchor plate 1316, but the head of the second screw type 1308 may not pass through the second hole or opening of the anchor plate 1316. In the configuration 13C, the head of the second screw type 1320 may sit above the top surface of the anchor plate 1316 but should not extend above the head of the first screw type 1304. The anchor plate 1316 helps to couple the two screws together as well as interface with the staple 1312.

While the first screw type 1304 is shown to have a larger shank diameter than the second screw type 1308 and 1320, it should be appreciated that such a configuration is not required. For instance, the first screw type 1304 and second screw type 1308 or 1320 may have the same shank diameters or the second screw type 1308 or 1320 may have a shank diameter that is greater than the shank diameter of the first screw type 1304.

FIG. 13B illustrates an embodiment where two screws of the same type are coupled to one another with the anchor plate 1316. In this particular embodiment, two screws of the first screw type 1304 are coupled to one another via the anchor plate 1316. Both screws 1304 may be configured to receive a cord 116, second cord 708, or the like.

FIG. 13D illustrates another embodiment where a first screw type 1304 is inserted through a staple 1332, but a different type of staple 1332 is used as compared to previous configurations. The staple 1332 is designed to interface with an anchor plate having an integrated tulip head 1324. The anchor plate with integrated tulip head 1324 may be axially fixed relative to the staple 1332 via a locking nut 1328. The locking nut 1328 may be used to tighten the anchor plate onto the top surface of the staple 1332. More specifically, the staple 1332 may include threads 1340 that interface with the locking nut 1328 and ribs 1336 that interface with the anchor plate. As the locking nut 1328 is tightened onto the threads 1340, the anchor plate with integrated tulip head 1324 may be pressed down onto the staple 1332. Ribs 1336 on the staple 1332 may help secure the anchor plate in rotation relative to the staple 1332.

In some embodiments, the first screw type 1304 may be inserted through the staple 1332 and anchor plate. In other embodiments, it may be suitable to use other screw types (e.g., a second screw type 1308 or third screw type 1320) in connection with the anchor plate having the integrated tulip head 1324 to achieve the embodiment illustrated in FIG. 13D.

FIG. 13E illustrates another embodiment where a looped cord 1012 is wrapped around the first screw type 1304. This particular configuration again shows the first screw type 1304 and second screw type 1308 coupled to one another via an anchor plate 1316. A locking nut 1328 is also shown to secure a looped cord 1012 onto a top surface of the anchor plate 1316.

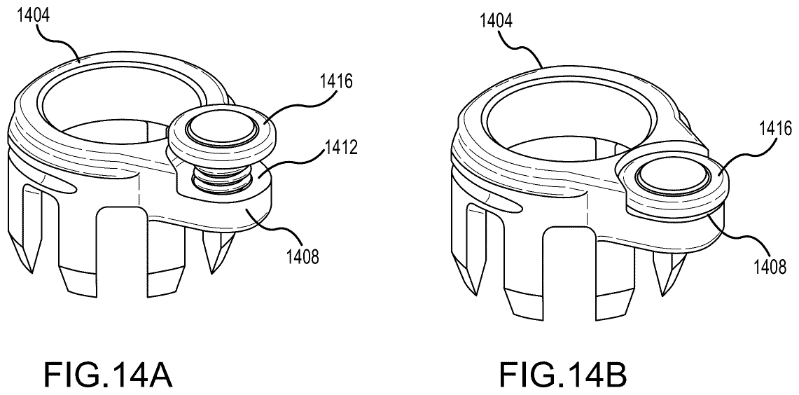

With reference now to FIGS. 14A and 14B, an improved staple 1404 will be described in accordance with at least some embodiments of the present disclosure. The staple 1404 is shown to include a feature that enables cooperation with a second cord 708 or looped cord 1012. In particular, the staple 1404 is shown to include an extension 1408 having a cord support face 1412. The extension 1408 may also include a threaded hole into which a post 1416 can be inserted and tightened.

A second cord 708 or looped cord 1012 may be configured to wrap around the shank of the post 1416 and then be pinched between a head of the post 1416 and the cord support face 1412. The extension 1408 may be integrally formed with the rest of the staple 1404. In some embodiments, the post 1416 may include a feature that prohibits the post 1416 from being completely removed from the extension 1408. In other words, the post 1416 may be a movable part of the staple 1404, but may not be configured to be removed from the staple 1404 unless unnecessary or undesired forces are placed thereon.

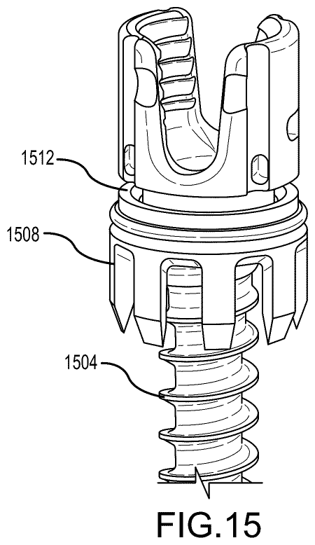

With reference now to FIG. 15, another example of a bone anchor will be described in accordance with at least some embodiments of the present disclosure. The bone anchor is shown to include a screw 1504 with a shank. The screw 1504 may also include a tulip head having a lower surface configured to interface with a second cord 708 or looped cord 1012. In some embodiments, the bone anchor may further include a staple 1508 that can receive the screw 1504 and that can secure a second cord 708 or looped cord 1012 between its cord support face 1512 and the tulip head of the screw 1504.

In some embodiments, the second cord 708 or looped cord 1012 may wrap around the head of screw 1504 and mate with the neck of the screw before the screw is fully seated. The second cord 708 or looped cord 1012 may then be tensioned, at which point the screw 1504 is fully seated (e.g., further driven into the bony structure and into the staple 1508). Fully screwing the screw 1504 into the staple 1508 may help to secure the second cord 708 or looped cord 1012 between the screw 1504 and staple 1508, thereby locking the second cord 708 or looped cord 1012 in place therebetween.

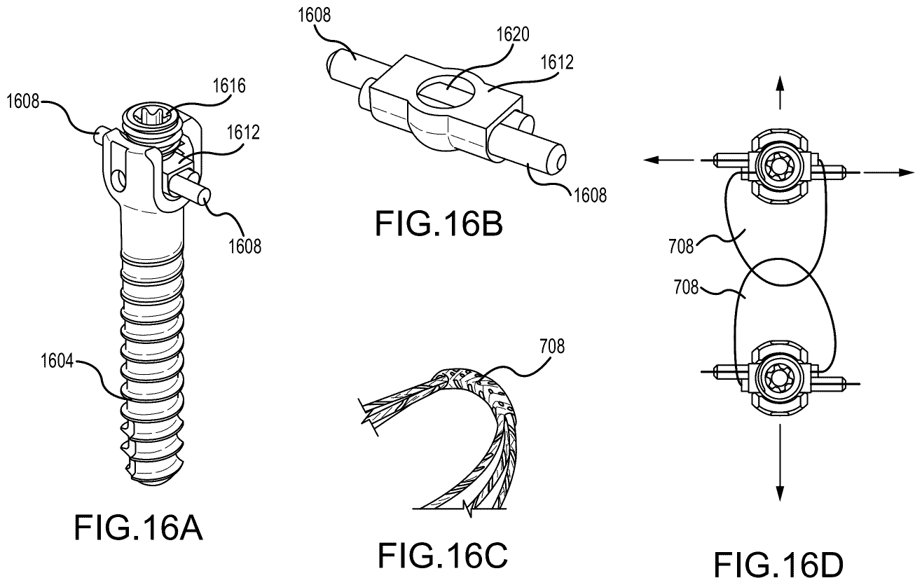

With reference now to FIGS. 16A-16D, additional details of coupling cords will be described in accordance with at least some embodiments of the present disclosure. FIG. 16B specifically illustrates a cord coupler 1612 that can be used to couple different cord portions 1608 to one another. The cord coupler 1612 can be configured to be received within a tulip head of a screw 1604. The cord coupler 1612 may also be configured to secure multiple cord portions 1608 together and to the screw 1604. The cord portions 1608 may correspond to independent pieces of a cord 116. Alternatively, or additionally, one cord portion 1608 may correspond to a cord 116 while another cord portion 1608 may correspond to a piece of a looped cord 1012 or to a second cord 708. Alternatively, or additionally, one cord portion 1608 may correspond to a piece of a looped cord 1012 or second cord 708 while another cord portion 1608 may correspond to another piece of a looped cord 1012 or second cord 708. Alternatively, or additionally, both cord portions 1608 may correspond to different sections of the same looped cord 1012 or second cord 708.

In the illustrated configuration, the cord coupler 1612 is shown to fit within the tulip head of the screw 1604 while also receiving both cord portions 1608. The cord coupler 1612 may include a two-piece plastic fitting that snaps together around two cord portions 1608. Once the cord coupler 1612 is positioned around the cord portions 1608, the cord coupler 1612 may be inserted into the tulip head of the screw 1604 and set therein with a set screw 1616.

The cord coupler 1612 may also include a hole 1620 on an upper surface thereof. The hole 1620 may provide access for the set screw 1616 to directly contact both cord portions 1608. The hole 1620 may be sized to expose at least half of each cord portion 1608 and to receive the bottom of the set screw 1616.

In some embodiments, the cord coupler 1612 provides an ability to daisy-chain multiple cords or second cords together. The cord portions 1608 can be locked into place at each screw 1604 with a set screw 1616. As shown in FIG. 16D, second cords 708 could be locked into screws 1604 which are rotated 90 degrees from standard configurations. Said another way, the passages in the tulip head of each screw 1604 may not be required to face one another. Rather, the passages in the tulip head of each screw 1604 may be directed orthogonally (or in some other non-facing arrangement) such that tightening of the loops against a side of the tulip head provides the desired tension to the screw 1604. This particular configuration may help to prohibit the cord 116 from slipping out of the tulip head of the screw (e.g., because the set screw 1616 loosened).

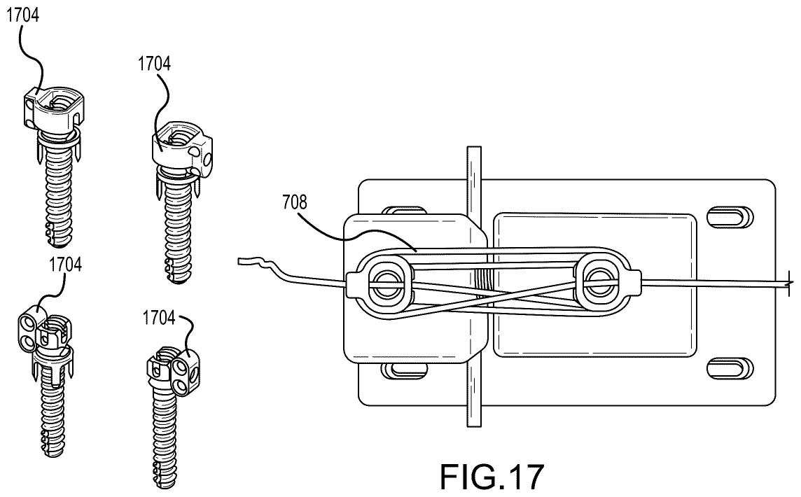

FIG. 16D also illustrates that the directionality of the cord portions 1608 within the screw 1604 are orthogonal to the direction of force/tension placed onto the head of the screw 1604. By utilizing a configuration of this type, it becomes possible to utilize stronger and redundant second cord 708 configurations. One example of a redundant second cord configuration is shown in FIG. 17. More specifically, primary cord could be routed into loops for supplemental strength and/or to correct spine thru tensioning. The routing of the cord could provide a mechanical advantage for tensioning. Screw caps 1704 could be designed so that cords are pre-routed and then dropped over screws for ease of insertion. Examples of such screw caps 1704 are shown in FIG. 17 as well.

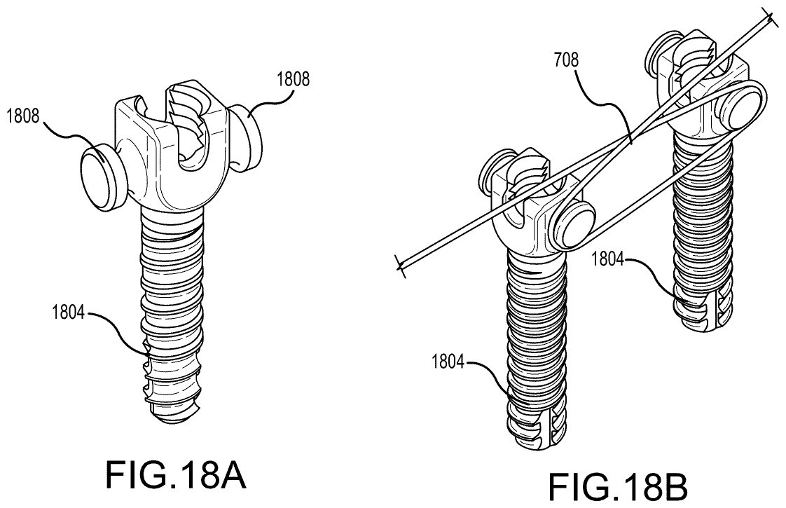

FIGS. 18A and 18B exhibit an alternative configuration of screw 1804 where a separate screw cap 1704 is not required. Instead, according to the examples of FIGS. 18A and 18B, the screw 1804 may be provided with one or more lateral posts 1808 on the head thereof. The lateral post(s) 1808 may function similarly to the screw caps 1704 in that the lateral post(s) 1808 support the looping of one or more second cords 708 onto the screw 1804. In some embodiments, a primary cord 116 or second cord 708 could be routed over the lateral posts 1808 in a way that provides a mechanical advantage for tensioning as well as supplemental fixation/strength. In the embodiment depicted in FIGS. 18A and 18B, the lateral posts 1808 may be oriented orthogonally to the channel passing through the tulip head of the screw 1804. Such a configuration allows the tensile forces applied to the screw 1804 by a second cord 708 to supplement a tensile force applied by a cord 116 fixed in the tulip head of the screw 1804.

Although the lateral post(s) 1808 are shown as being integrally formed with the screw 1804, it should be appreciated that the lateral post(s) 1808 may also be modular. In other words, the lateral post(s) 1808 may be provided as a modular component, similar to the screw caps 1704 illustrated in FIG. 17.

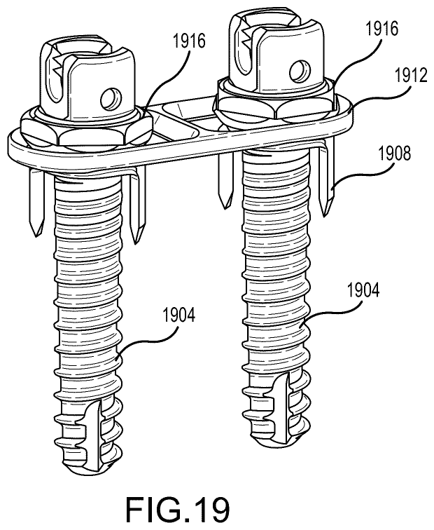

With reference now to FIG. 19, yet another example of a bone anchor or system of bone anchors will be described in accordance with at least some embodiments of the present disclosure. The bone anchors are illustrated to include two or more screws 1904 that are coupled to one another with an anchor plate 1912. The anchor plate 1912 is similar to other anchor plates depicted and described herein except that the anchor plate 1912 includes holes therein that are substantially larger in one dimension than the outer diameter of the screws 1904. Such a configuration allows the screws 1904 to be inserted and then the position of the anchor plate 1912 can be adjusted (e.g., slid, translated, etc.) relative to one or both screws 1904. This configuration also affords some flexibility when connecting two screws 1904.

The configuration of FIG. 19 also provides an ability to rigidly connect (e.g., fuse) two screws 1904. In some embodiments, the screws 1904 may accept the anchor plate 1912 to support a hybrid (e.g., fusion+tether construct) or revision. The specific level with the plate 1912 would be fused but a single primary cord 116 could pass over the length of the construct (e.g., between the screws 1904). In some embodiments, spherical or cylindrical features on the bottom of the locking nut 1916 may interface with the slot-like channel of the anchor plate 1912. As the locking nut 1916 is tightened, the anchor plate 1912 may be pressed against a top surface of the staple 1908, but the rounded feature on the bottom of the locking nut 1916 may allow for the screws to sit at varying angles. In other words, the rounded bottom of the locking nut 1916 may allow the screws 1904 to have some substantial separation from one another and may avoid the requirement that both screws 1904 be co-planar with one another. In particular, one of the screws 1904 may be provided at an angle other than parallel with the other screw 1904 thanks to the rounded bottom of the locking nut 1916.

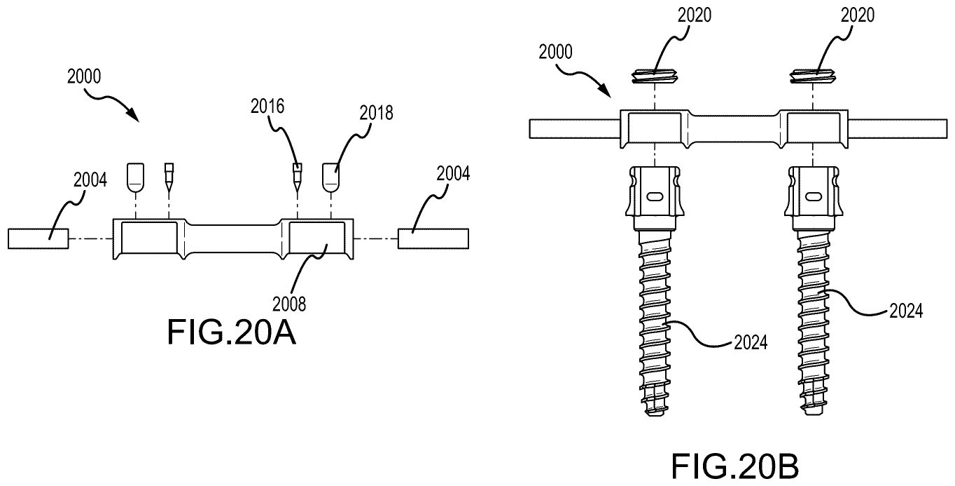

With reference now to FIGS. 20A and 20B, an alternative configuration of a cord coupler 2008 will be described in accordance with at least some embodiments of the present disclosure. The configuration illustrated in these figures enables a cord-rod connector (e.g., cord coupler 2008) that allows two different cord portions 2004 to be coupled together as part of the stabilization system 2000. Rather than coupling the cord portions 2004 at the same screw as shown in previous figures, this particular embodiment utilizes the cord coupler 2008 to couple one cord portion 2004 above one screw 2024 and to couple another cord portion 2004 above another screw 2024. In this configuration, adjacent screws 2024 can be coupled with the cord coupler 2008.

In some embodiments, the cord coupler 2008 may be constructed of a metal or metallic alloy. In some embodiments, the cord coupler 2008 may be constructed of a flexibly-resilient material such as a polymer, rubber, or the like. The cord coupler 2008 may also include notches that align and fit within the tulip head of a screw 2024. The cord coupler 2008 may be fixed within a screw 2024 with a set screw 2020 that engages threading in the tulip head of the screw 2024.

The cord coupler 2008 may also include one or more cord-securing members to connect the cord portion 2004 to the cord coupler 2008. In some embodiments, the cord-securing member(s) may include a needle 2016. In some embodiments, the cord-securing member(s) may include a pin 2018 (e.g., a blunt-tipped element) that secures the cord portion 2004 into the cord coupler 2008. Alternatively, or additionally, the cord portion(s) 2004 may be secured in the cord coupler 2008 with the set screws 2020, meaning that a cord-securing member may not be necessary.

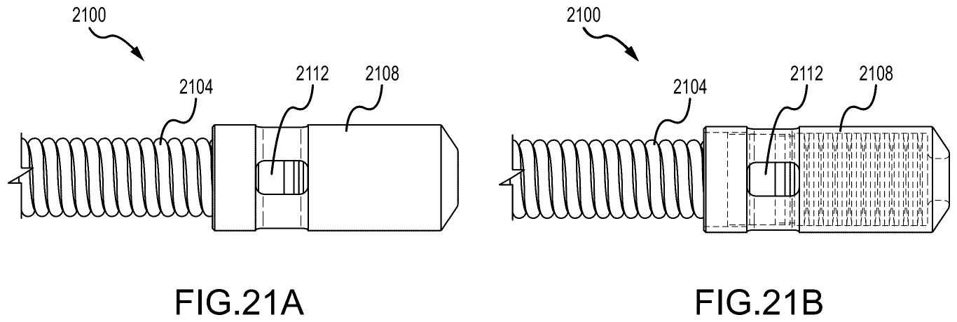

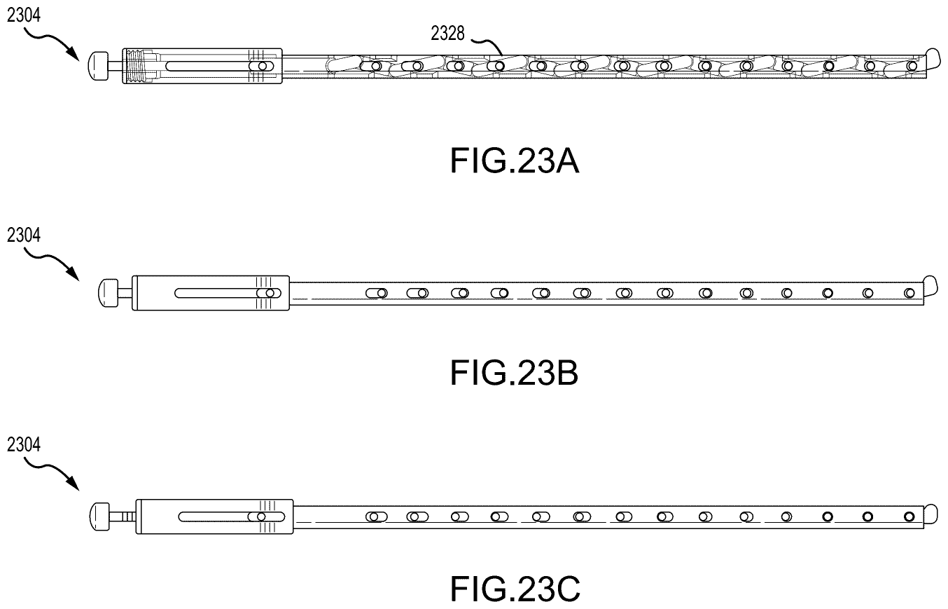

Referring now to FIGS. 21A-27B, details of an instrument and components thereof that may be used during a tensioning step. It should be appreciated, however, that the various techniques and devices could also be used during other surgical steps (e.g., to implant one or more bone anchors, tension a cord, tension a second cord, or otherwise implant the tensioning system). More specifically, and as will be described in further detail herein, a surgical instrument may be provided to implant the bone anchors and to apply tension to a cord 116, looped cord 1012, or second cord 708. The instrument may be referred to as a tensioner, counter tensioner, or tensioning instrument. Moreover, while various components of an instrument will be described herein, it should be appreciated that various components may be omitted or exchanged with other components.

FIGS. 21A and 21B illustrate a tensioning tip 2100. The tensioning tip 2100 is illustrated as a two-piece design with stacked washers (e.g., Belleville washers) placed between the pieces. The two pieces may include a housing 2108 and spring 2104. Although spring 2104 is illustrated and described as having a particular configuration, it should be appreciated that other devices or components could be used. For instance, the spring 2104 may include or be replaced with any suitable flexible tubing with a lumen running through it large enough to accommodate cord 116.

The housing 2108 may include a window 2112 that allows a relative motion between the housing 2108 and spring 2104 to be indicated. The cord 116 may be allowed to pass through the center of the tensioning tip 2100 (e.g., passing through the lumen of the spring 2104 and a hole in the tip of the housing 2108). The end of the housing 2108 may interface with a counter tensioner 2200 as will be described in further detail.

More specifically, compression of stacked washers will indicate (through an indicator shown in the window 2112) the amount of tension applied to a cord 116. As the tensioning tip 2100 is pressed against the counter tensioner 2200, an indicator may move laterally within the window 2112.

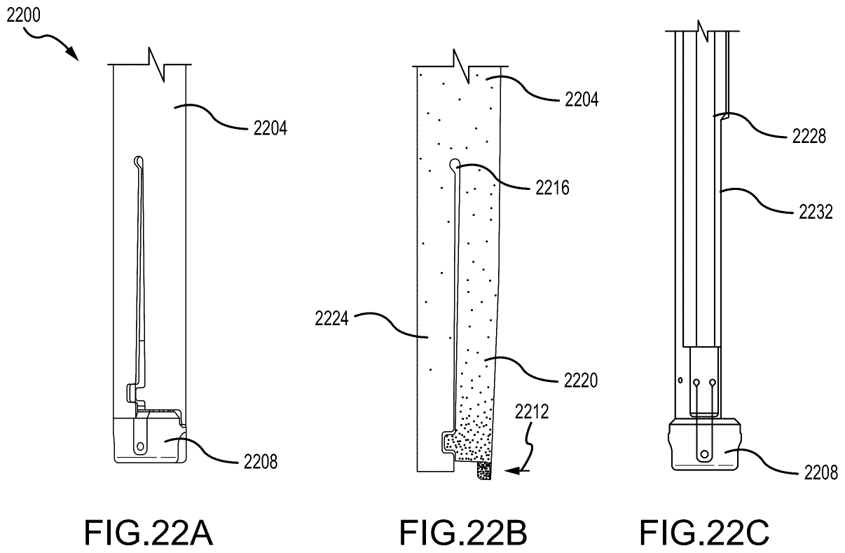

The counter tensioner 2200 may also include a number of components to help apply tension to a cord 116 while securing to a bone anchor (e.g., to pull a cord 116 through a bone anchor prior to fixing the cord 116 within the bone anchor). FIGS. 22A-25 illustrate details of the components of the counter tensioner 2200. In some embodiments, the counter tensioner 2200 comprises an outer sleeve 2204 and inner sleeve 2208. The outer sleeve 2204 may include a slot cut therein to separate a bottom portion of the outer sleeve 2204 into a first arm 2220 and second arm 2224. The first arm 2220 and second arm 2224 may meet at a slot apex 2216, that includes an enlarged feature to facilitate relative movement of the arms 2220, 2224. As a force 2212 is applied against the first arm 2220 during cord 116 tensioning, the first arm 2220 may deflect toward the second arm 2224. In some embodiments, the force 2212 is applied against the first arm 2220 by the tensioning tip 2100 when the tensioning tip 2100 is connected to the cord 116.

The inner sleeve 2208 is shown to include a passage 2228 for additional components of the counter tensioner 2200, The passage 2228 may include a cut 2232 to provide clearance for compression of the outer sleeve 2204. Said another way, the cut 2232 may be dimensioned to enable deflection of the first arm 2220 when the outer sleeve 2204 is positioned over the inner sleeve 2208 as shown in FIG. 22A.

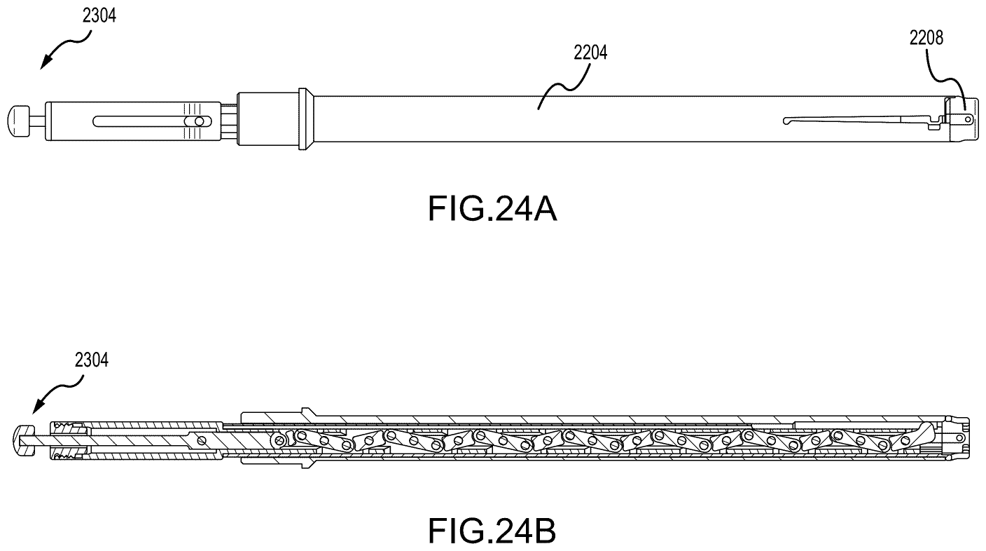

FIGS. 24A and 24B illustrate additional details of the counter tensioner 2200 with a pivot ladder 2304 inserted therein. The pivot ladder 2304 may extend through the passage 2228 of the inner sleeve 2208 to a point where the end of the pivot ladder 2304 contacts the end of the inner sleeve 2208. The pivot ladder 2304 may also include an outer sleeve, having an outer diameter less than an inner diameter of the passage 2228. The pivot ladder 2304 may further include a series of pivoting linkages each which rotate about a center pin. The subsequent pins ride in a slot along the centerline of the linkages. In such a configuration, lateral displacement of each linkage is additive. Therefore, while there may be a small amount of movement at the tip (˜2 mm), the last linkage can move ˜5 mm or more, depending upon the geometry used for the pivot ladder 2304.

In some embodiments, a piston and spring are provided at the distal end of the instrument pivot ladder 2304. The piston and spring may push on the linkages so that they sit in a rotated state, until force is applied at the tip by force 2212. The spring piston provides space for a spring and also markings to indicate displacement and how it relates to force (e.g., tension) on the counter tensioner's 2200 deflection beam (e.g., the deflection of the first arm 2220 under application of force 2212).

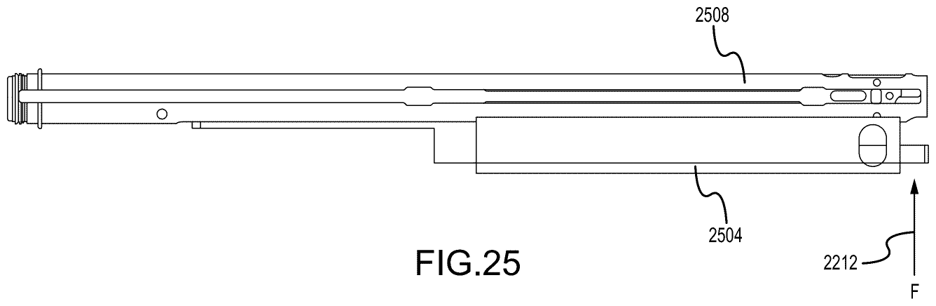

An alternative configuration of the counter tensioner 2508 is shown in FIG. 25 where it can be seen that the deflection beam 2504 is positioned external to the main channel of the inner sleeve 2208. In particular, rather than using an outer sleeve 2204 as shown in FIGS. 22A and 22B, a deflection beam 2504 is positioned on a component part that attaches to a side of the instrument 2508. In this configuration, the deflection beam 2504 may flex across some or the entire length of the deflection beam 2504. A force 2212 will be applied to the deflection beam 2504 as tension is applied to the cord 116 passing through the tip of the instrument 2508, while the instrument 2508 holds or is secured to a bone anchor.

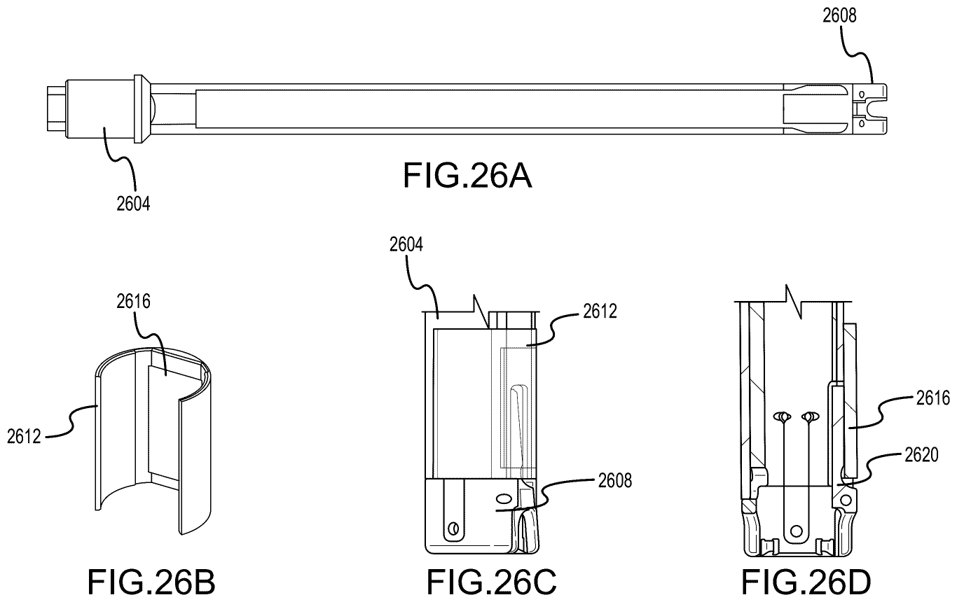

FIGS. 26A-26D illustrate another configuration of the counter tensioner 2604 where a force sensor 2616 (which may also be provided as a pressure sensor, displacement sensor, or the like) is provided at a distal end of the counter tensioner 2604. The counter tensioner 2604 may also include an anchor interface 2608 for releasably attaching to a bone anchor when the bone anchor is secured to a bony structure. Like other instruments described herein, the counter tensioner 2604 may be configured to pass through an MIS tube or similar delivery device to facilitate a minimally invasive surgical procedure.

The force sensor 2616 may include an outer sleeve 2612 that is secured to an outer portion of the counter tensioner at the distal end (e.g., near the anchor interface 2608). The counter tensioner 2604 may also include a pivoting member 2620 that contacts the force sensor 2616. Pivoting member 2620 is actuated by the force 2212, which contacts and causes compression of the force sensor 2616. In some embodiments, the force sensor 2616 may be calibrated to display an amount of force 2212 at the anchor interface 2608, and provide a digital readout of the same. In other words, the sensor 2616 may be configured to communicate a force reading via a wired or wireless signal (e.g., via an antenna) to a user interface device provided at a proximal end of the counter tensioner 2604.

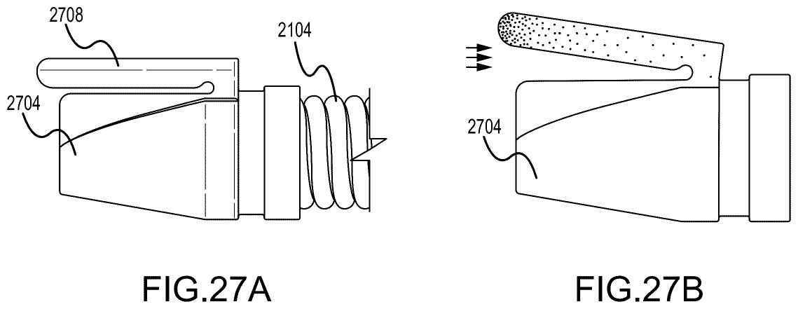

As can be seen in FIGS. 27A and 27B, the tensioning tip 2100 may be modified to include additional or alternative features for measuring or interpreting forces applied by (or to) the tensioning tip 2100. Specifically, but without limitation, the tensioning tip 2100 may include a deflection beam 2708 that is offset from the slot in which the cord 116 passes. The deflection beam 2708 may be integrally formed with the housing 2704. The deflection beam 2708 may interface with an outer diameter of a counter tensioner 2200, 2508, 2604. In some embodiments, as forces are applied against the deflection beam 2708, the deflection beam 2708 will displace (e.g., move outwardly). Displacement of the deflection beam 2708 may indicate an amount of tension that is being pulled on the cord 116 by the tensioning tip 2100. Like other configurations of the tensioning tip 2100, the cord 116 may still be configured to pass through the center of the tensioning tip 2100 such that the tensioning tip 2100 interfaces with the counter tensioner as tensile forces are applied to the cord 116.

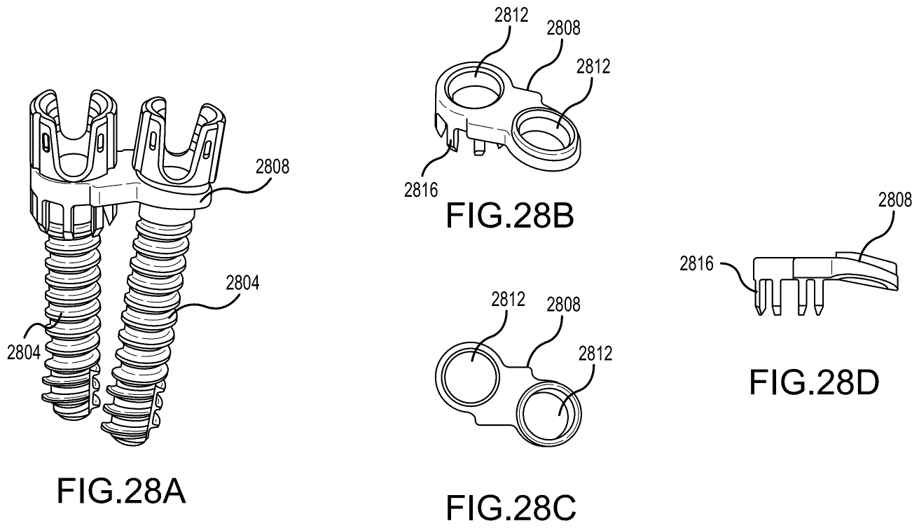

Referring now to FIGS. 28A-28D, another example of a bone anchor will be described in accordance with at least some embodiments of the present disclosure. The bone anchor is similar to at least some other bone anchors depicted and described herein, in that the bone anchor may include two or more screws 2804 that are coupled via an anchor plate 2808. The anchor plate 2808 is shown to include two non-planar openings 2812. Each non-planar opening 2812 of the anchor plate 2808 is sized to receive a screw 2804. The inner diameter of each non-planar opening 2812 may be greater than an outer diameter of the screw 2804 but less than a diameter of the shoulder of the tulip head of the screw 2804.

The anchor plate 2808 may have a width that enables the anchor plate 2808 to be delivered through an MIS tube. In some embodiments, the anchor plate 2808 may comprise a width of no more than 15 mm, thereby enabling the anchor plate 2808 to be delivered through an MIS tube of 15 mm. The anchor plate 2808 may then be flipped or rotated at the distal end of the MIS tube and then pressed into the bony structure before screws 2804 are delivered thereto.

As can be seen in FIG. 28D, one of the openings 2812 may be provided in a first plane while another of the openings 2812 may be provided in a second plane that is angled at more than zero degrees relative to the first plane. The anchor plate 2808 may comprise a rigid member that substantially fixes the orientation of the first plane and the second plane. In other embodiments, the anchor plate 2808 may comprise a flexible material (e.g., plastic) that allows some amount of bending to occur between the openings 2812.

The anchor plate 2808 is also depicted to include an integral staple. More specifically, one or both of the openings 2812 may be provided with one or more staple arms 2816 that extend downwardly from the opening 2812. The arms 2816 may be configured to press into a bony structure to at least partially secure the anchor plate 2808 to the bony structure while screws 2804 are driven through the anchor plate 2808 and into the bony structure. In some embodiments, the openings 2812 may be tilted relative to each other in at least two dimensions such that screws 2804 extending through both opening 2812 do not intersect or collide with one another.

The bone anchor of FIGS. 28A-28D may be configured to support a two-cord configuration. Said another way, each screw 2804 may be provided with a tulip head to receive a cord 116, second cord 708, or a combination thereof. It may be possible, however, to utilize a screw of a different type (e.g., not both screws in the bone anchor need to be provided with tulip heads).

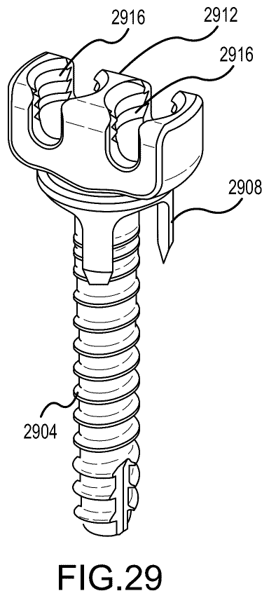

With reference now to FIG. 29, another example of a bone anchor that supports a two-cord configuration will be described in accordance with at least some embodiments of the present disclosure. The bone anchor is shown to include a screw 2904 and staple 2908. The screw 2904 is further shown to include a dual-slot head 2912 in which two cord slots 2916 are provided. In some embodiments, the dual-slot head 2912 is integrally formed with the shank of the screw 2904 (e.g., the screw 2904 and dual-slot head 2912 are a single piece of material). In some embodiments, the dual-slot head 2912 may correspond to a modular element that is attachable to a top of the screw 2904.

A bottom surface of the dual-slot head 2912 may include one or more ribs that interface with one or more ribs on a top surface of the staple 2908. The ribs may help to substantially preclude rotation of the dual-slot head 2912 relative to the staple 2908 (and therefore the bony structure). In some embodiments, the staple 2908 may first be inserted into the bony structure and then the screw 2904 may be driven through the staple 2908. Once the ribs of the staple 2908 interface corresponding ribs of the dual-slot head 2912, the screw 2904 may be prohibited from further rotation and insertion.

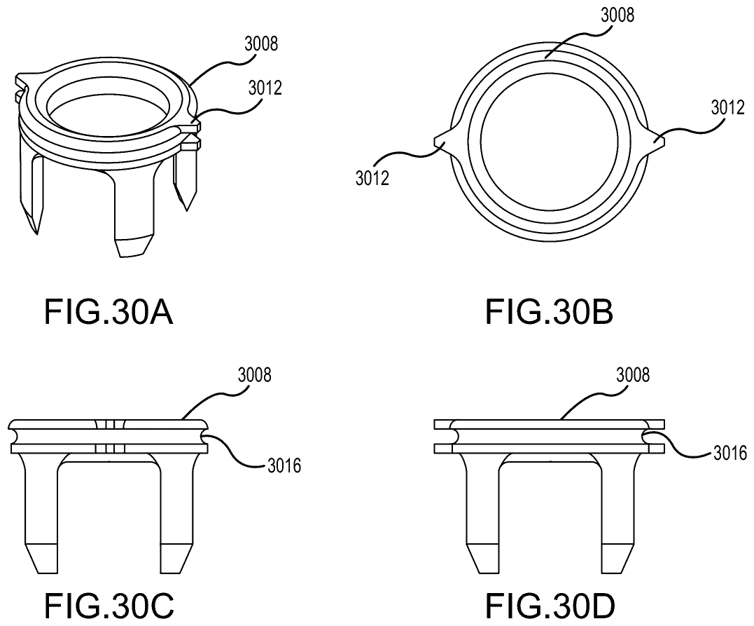

With reference now to FIGS. 30A-30D, additional details of staple 3008 having an alignment feature 3012 will be described in accordance with at least some embodiments of the present disclosure. The staple 3008 may be similar to other staples depicted and described herein. Moreover, the staple 3008 may be used with any of the bone anchors depicted and described herein.

In some embodiments, the staple 3008 may include one or more arms and an alignment feature 3012. The arms may be configured to pierce and hold a bony structure. The alignment feature 3012 may include one or more features of the staple 3008 that are visible from above (see FIG. 30B) and that help determine an orientation of the staple 3008 when being inserted from above.

The staple 3008 may further include a cord receiver 3016 that is similar to other cord receivers of other staples depicted and described herein. The cord receiver 3016 may be configured to receive a cord 116, a looped cord 1012, or a second cord 708. In some embodiments, the alignment feature 3012 may be provided as a rotatable sleeve that is configured to rotate relative to the inner portion of the staple 3008 that includes the arms. In other words, the alignment feature 3012 may be freely rotatable relative to other portions of the staple 3008. During implantation, a surgeon may place a ring having the alignment feature 3012 over the top of the base portion of the staple 3008. After the ring is placed on the base portion of the staple 3008, the surgeon can rotate the ring and its alignment features relative to the rest of the staple 3008. This freedom of rotation may allow the surgeon to align the ring as desired, then use the alignment features 3012 to align screws inserted through the staple 3008. For example, the surgeon may insert a screw into the staple 3008 until a feature of the screw (e.g., a post, a tulip head, a cord receiver, etc.) is aligned with the alignment feature 3012.

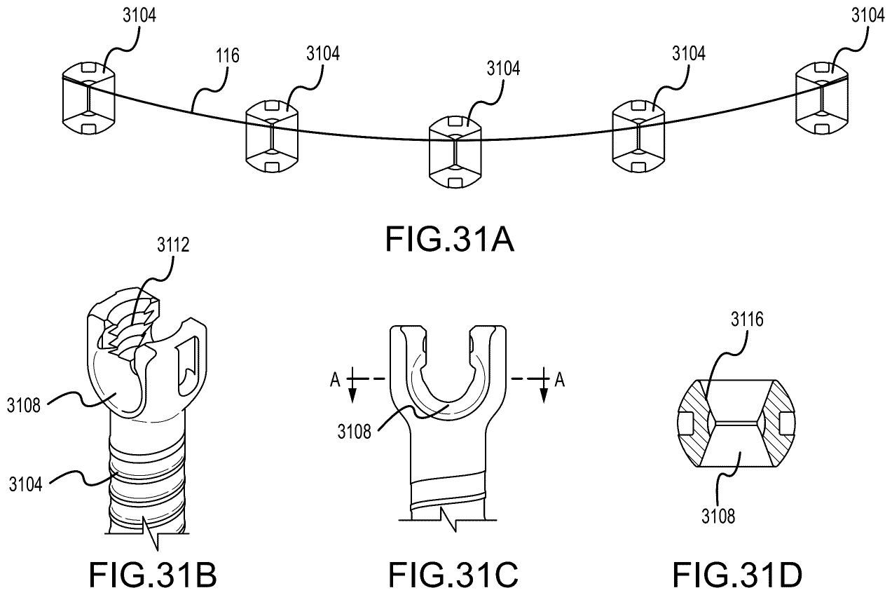

Referring now to FIGS. 31A-31D, additional details of a system having a plurality of bone anchors coupled with a cord 116 will be described in accordance with at least some embodiments of the present disclosure. The system may be provided as an implanted construct that helps to apply tensile forces between multiple vertebrae, in which the screws 3104 are implanted.

In some embodiments, one or more screws 3104 may include an angled sidewall 3116 in the tulip head. More specifically, the tulip head of the screws 3104 may include a cord receiver 3108 and threading 3112 for a set screw, like many of the other screws depicted and described herein. This particular screw 3104 may also include at least one angled sidewall that forms part of the cord receiver 3108. The angled sidewall 3116 may have an hourglass shape that allows the cord 116 to follow a natural curvature without creating contact stress between the cord 116 and cord receiver 3108.

In other words, by tapering the entry way into the cord receiver 3108, the angled sidewall helps minimize pinching or other stresses that might cause damage to a cord 116, especially as the cord 116 is placed under tension and secured in the tulip head. FIG. 31D, which corresponds to a cross-sectional view along line A-A, helps to illustrate the hourglass figure of the angled sidewall 3116 at the center of the cord receiver 3108. FIG. 31B helps illustrate that the angled sidewall 3116 creates a tapered threading 3112 into the screw 3104, meaning that more threading exists higher up on the screw.

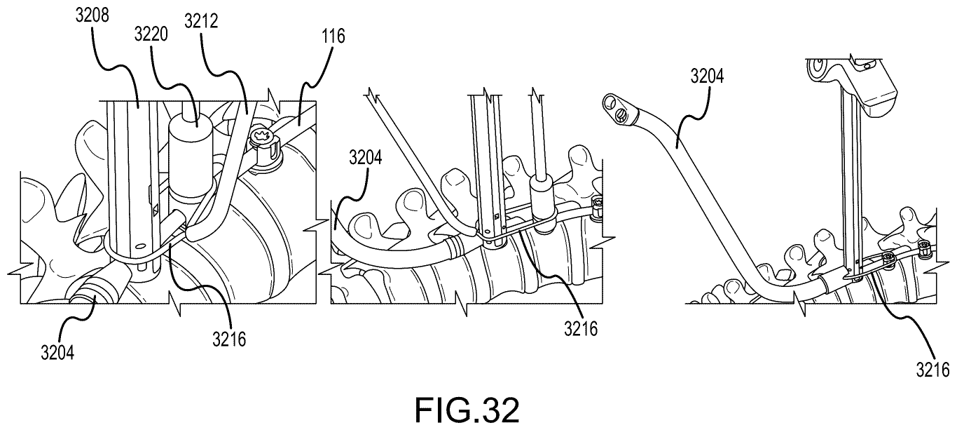

With reference now to FIGS. 32-36C, additional systems and methods for applying tension to a second cord 3216 between two or more bone anchors will be described in accordance with at least some embodiments of the present disclosure. FIG. 32 illustrates an example of a system and the components thereof that can be used to place and utilize a second cord 3216. The system is shown to include a snake 3204, a tensioning tower 3208, a screw attachment tower 3220, and a second cord placement instrument 3212. Each of the instruments depicted as part of the system may help facilitate MIS procedures for the placement and tensioning of the second cord 3216. The second cord 3216 may be similar or identical to other second cords or looped cords depicted and described herein.

In some embodiments, the second cord 3216 may be looped around both a tensioning tower 3208 and the screw attachment tower 3220. Both the tensioning tower 3208 and screw attachment tower 3220 may be coupled with a bone anchor (e.g., a top of a screw) prior to positioning the second cord 3216. The snake 3204 may be used to apply a tension to the cord 116 traversing the bone anchors. In some examples, the snake 3204 may correspond to an example of the tensioning tip and the tensioning tower 3208 may correspond to an example of the counter tensioner. In other words, the snake 3204 may include the spring and housing assembly that allows the cord 116 to pass therethrough. The tensioning tower 3208 may be designed to interact with the snake 3204 to apply tension to the cord 116 prior to locking the cord in the bone anchors with set screws.

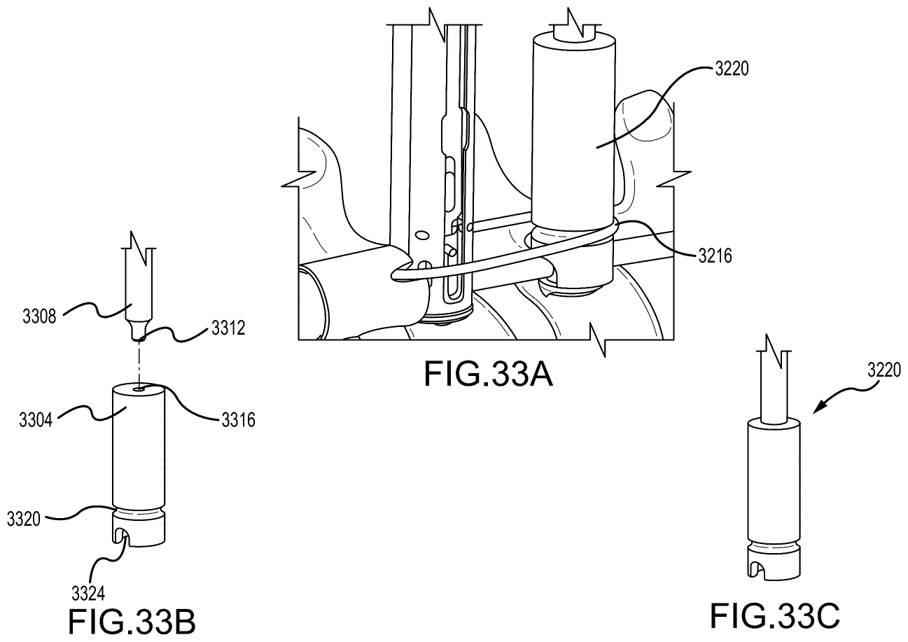

Additional details of the screw attachment tower 3220 are depicted in FIGS. 33A-33C. In the illustrated but non-limiting examples, the screw attachment tower 3220 may include a tower body 3304 and an inserter 3308. The tower body 3304 may include a recess 3316 that cooperates with a coupling pin 3312 of the inserter 3308. The tower body 3304 may also include a second cord notch 3320 and a cord notch 3324. The second cord notch 3320 may be configured to retain the second cord 3216 and substantially prohibit the second cord 3216 from sliding axially along the tower body 3304.

The cord notch 3324 may provide a passageway for the cord 116 to pass underneath the screw attachment tower 3220. The tower body 3304 may further include features for interfacing with a top of a bone anchor (e.g., to retain a connection with a top of a bone anchor or screw) while tension is applied to the second cord 3216 and/or cord 116. In some embodiments, the screw attachment tower 3220 may set upon a top of a bone anchor. In some embodiments, the screw attachment tower 3220 may releasably connect to a top of the bone anchor, thereby substantially prohibiting axial movement relative to the bone anchor until released. The two-piece configuration of the screw attachment tower 3220 may allow the instrument to remain in place without requiring use of a separate port/incision.

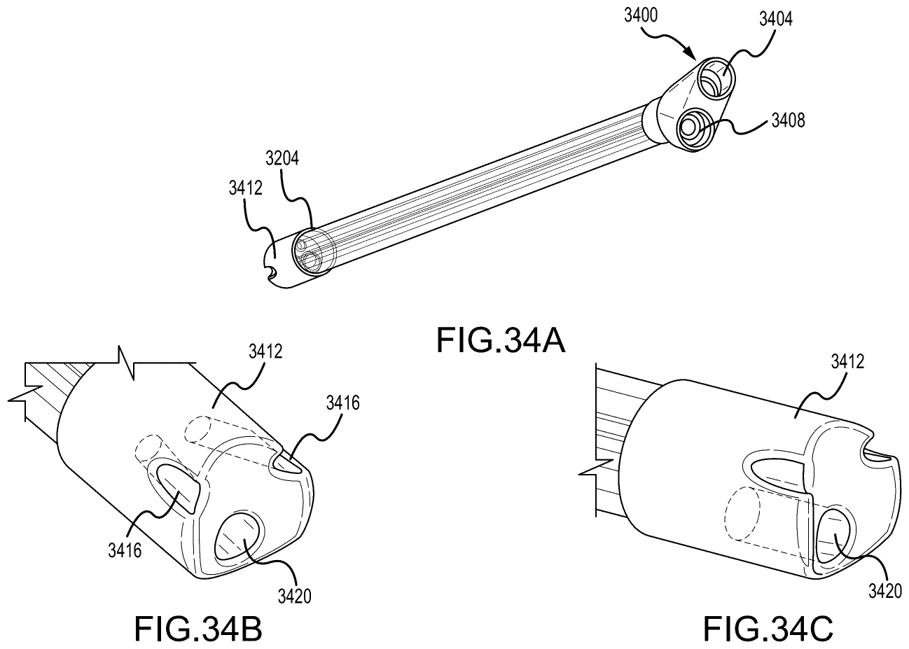

FIGS. 34A-36C illustrate additional details of the snake 3204 according to at least some embodiments. The snake 3204 is shown to include a distal end 3412 and a proximal end 3400. The distal end 3412 may be configured to interface with a distal end of the tensioning tower 3208. In some embodiments, the distal end 3412 of the snake 3204 may have a tensioning tip extending therefrom to support tensioning of a cord 116.

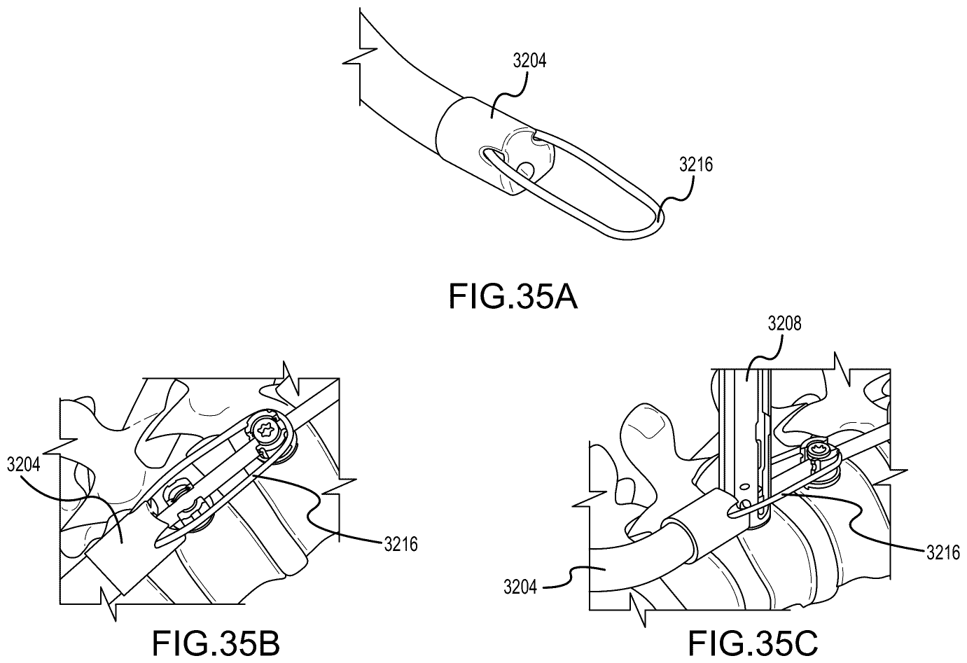

As shown in FIGS. 34B and 34C, the distal end 3412 may include a primary cord passage 3420 and two or more second cord passages 3416. The primary cord passage 3420 may be connected with a first opening 3404 at the proximal end via a primary passage lumen 3620 while the two or more second cord passages 3416 may be connected with the second opening 3408 via one or more secondary passage lumen 3616. In some embodiments, as shown in FIGS. 35A-35C, the second cord 3216 may have one portion passing through one of the second cord passages 3416 and another portion passing through the other of the second cord passages 3416. Free ends of the second cord 3216 may be accessible via the second opening 3408 and can be pulled to apply tension on bone anchors via the second cord 3216. The primary cord passage 3420 and lumen 3620 may be sized to allow a cord 116 to pass therethrough. In some embodiments, a diameter of the primary cord passage 3420 and lumen 3620 may be greater than a diameter of either second cord passage 3416.

By providing independent passages and lumen through the snake 3204, different tensions can be applied to the cord 116 as compared to the second cord 3216. Additionally, separate tensioning guns 3604 can be used independently and possibly simultaneously to apply different tensile forces to adjacent bone anchors via the second cord 3216 and the cord 116. In some embodiments, the second cord 3216 may extend from the distal end 3412 of the snake 3204 as a loop. The loop can be extended and manipulated over the bone anchor(s) at the adjacent level(s). The second cord 3216 may interface with features on the bone anchor (or an additional instrument placed on the bone anchor, such as an attachment tower 3220) and is retained on these features while under tension.

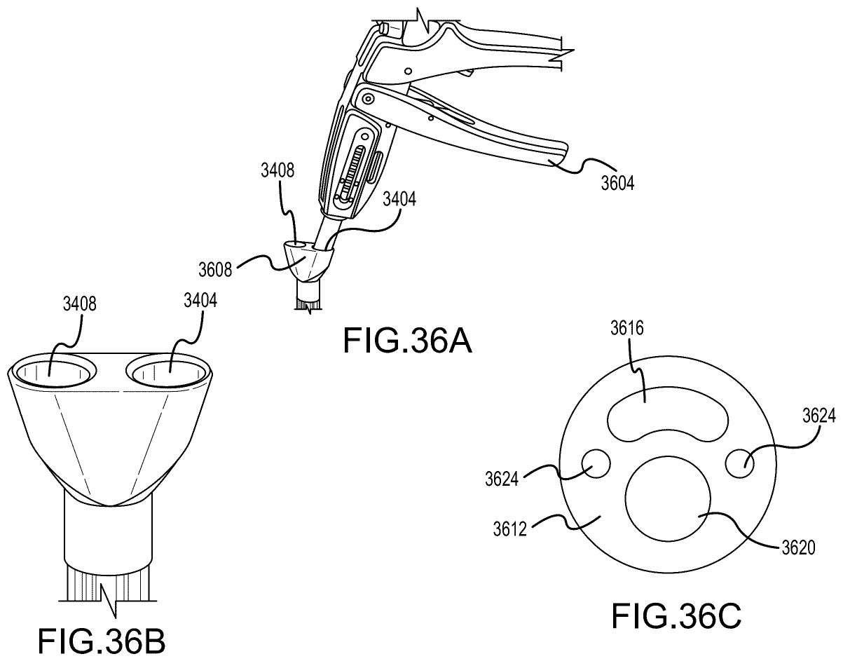

FIG. 36C illustrates a cross-sectional view in a middle portion of the snake 3204. As shown in FIG. 36C, the snake body 3612 may include multiple lumens through which different surgical elements can pass. The primary passage lumen 3620 may allow the cord 116 to pass therethrough, the secondary passage lumen 3616 may allow the second cord 3216 to pass therethrough, other lumen 3624 may permit other implants, cords or instruments to pass therethrough.

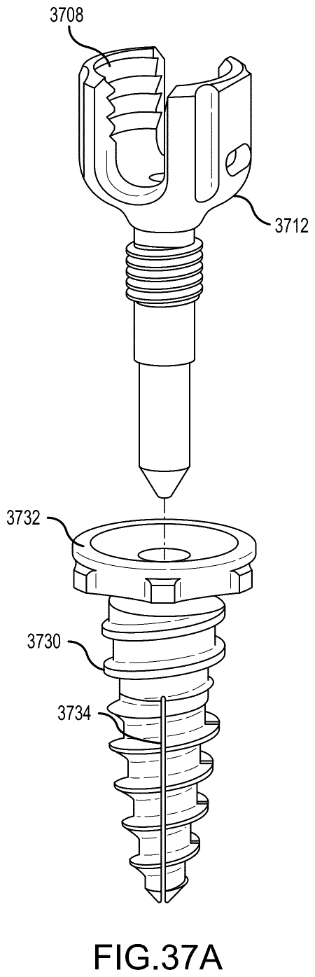

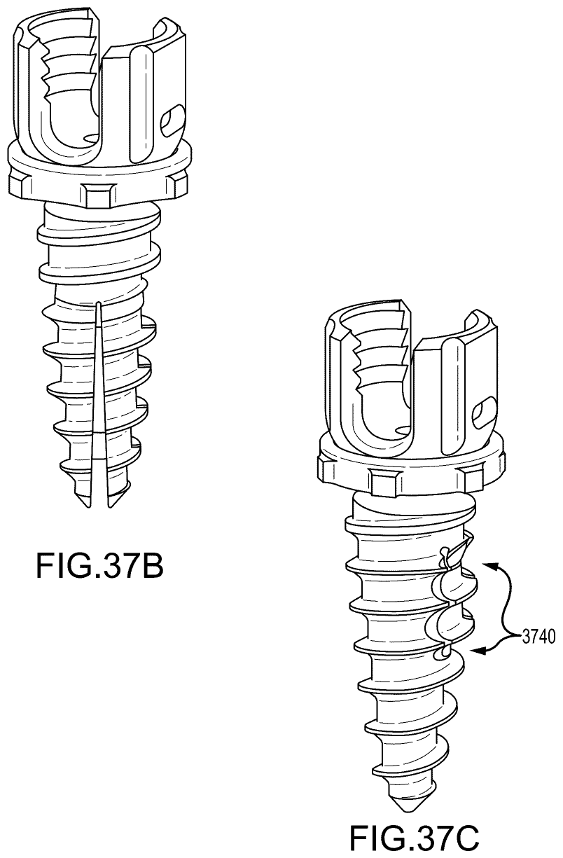

FIGS. 37A-37C illustrate a bone anchor 3712 that includes a screw 3708 with a cord receiving head with internal threads capable of receiving a set screw 204. The bone anchor 3712 further comprises an anchor cone implant 3730 that comprises instrument engagement features 3732 at the wider, proximal end of the anchor cone implant 3730. The instrument engagement features 3732 may comprise notches, slots, grooves, ridges, or other features that enable an instrument to engage with the anchor cone implant 3730 in a manner that enables the instrument to thread or screw 3708 the anchor cone implant 3730 into a vertebral body 104. A pilot hole may be predrilled at the location the anchor cone implant 3730 is to be implanted.

The anchor cone implant 3730 may be fabricated of titanium, tantalum or other biocompatible metal or other biocompatible material that is sufficiently capable of being screwed into bone. The anchor cone implant 3730 may further include a split 3734 at the tip end of the cone. The split 3734 may be fabricated by laser cutting the split or by means of cutting with a diamond tip saw or other know cutting methods. The split 3734 is completely through the tip of the cone and goes up the cone a sufficient distance that when the screw 3708 is threaded into the anchor cone implant 3720, the tip of the cone is forced to splay outward. The anchor cone implant may further include one or more U-shaped cuts 3740 in a side wall of anchor cone implant 3730. The U-shaped cuts extend through the side wall of the anchor cone implant 3730, such that when the screw 3708 is threaded into the anchor cone implant 3730, the U-shaped cut areas of the sidewall will splay away from the sidewall of the anchor cone implant 3730.

The bone anchor 3712 may be implanted by creating a pilot hole into a vertebral body 104; utilizing an instrument to engage instrument engagement features 3732 on the head of the anchor cone implant 3730; threading the anchor cone implant 3730 into the pilot hole in the vertebral body 104; engaging engagement features on the tulip head of the screw 3708 with an instrument; threading the screw 3708 into a central threaded lumen in the anchor cone implant 3730; inserting a cord 116 into the cord receiver of the screw 3708; tensioning the cord 116 with a cord tensioner; and threading a set screw 204 into the internal threads of the cord receiver of the screw 3708. A bone anchor 3712 that includes an anchor cone implant 3730 may be used to improve bone purchase or mitigate screw migration. A bone anchor 3712 that includes an anchor cone implant 3730 may be utilized when a revision surgery is necessary or if the bone quality is poor.

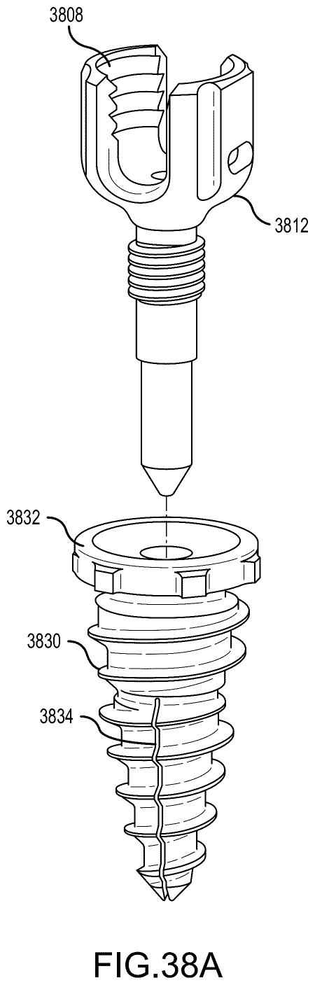

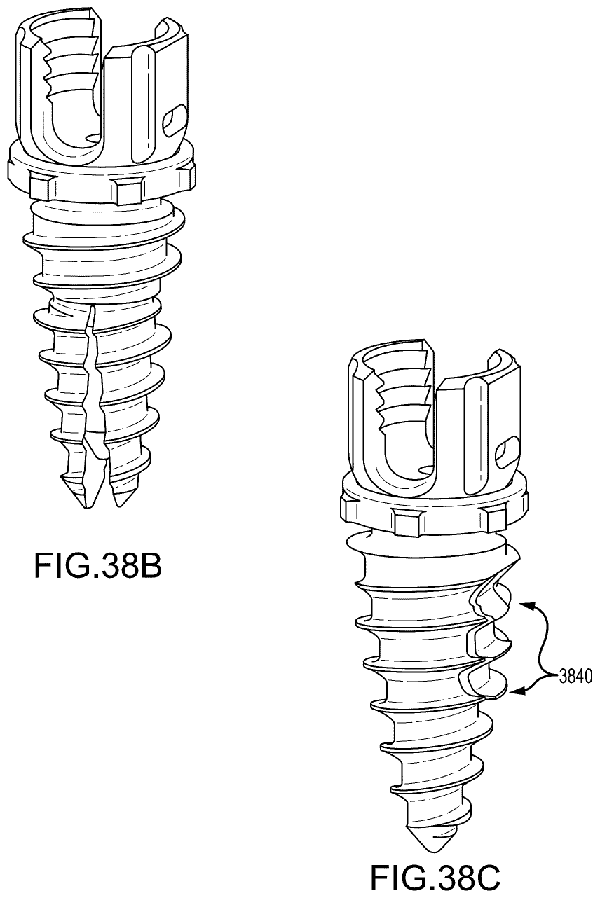

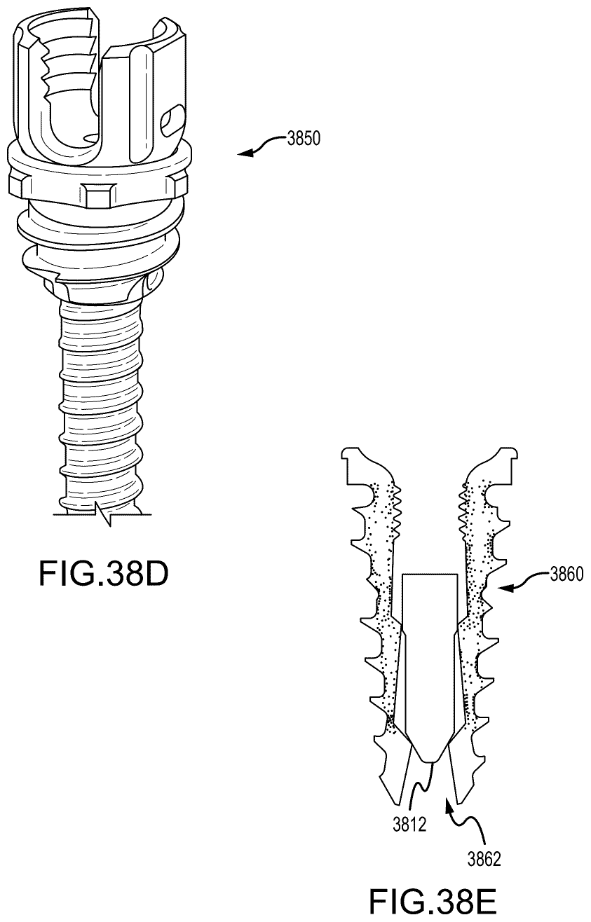

FIGS. 38A-38C illustrate a bone anchor 3812 that includes a screw 3808 with a cord receiving head with internal threads capable of receiving a set screw 204 (not shown). The bone anchor 3812 further comprises an anchor cone implant 3830 that comprises instrument engagement features 3832 at the wider, proximal end of the anchor cone implant 3830. The instrument engagement features 3832 may comprise notches, slots, grooves, ridges, or other features that enable an instrument to engage with the anchor cone implant 3830 in a manner that enables the instrument to thread or screw the anchor cone implant 3830 into a vertebral body 104. Additionally, a pilot hole may be predrilled at a predetermined location the anchor cone implant 3830 is to be implanted.

The anchor cone implant 3830 may be fabricated of titanium, tantalum or other biocompatible metal or other biocompatible material that is sufficiently capable of being screwed into bone. The anchor cone implant 3830 may further include a split 3834 at the tip end of the conc. The split 3834 may be fabricated by laser cutting the split or by means of cutting with a diamond tip saw or other know cutting methods. The split 3834 is completely through the tip of the cone and goes up the cone a sufficient distance that when the screw 3808 is threaded into the anchor cone implant 3830, the tip of the cone is forced to splay outward. The anchor cone implant 3830 may further include one or more U-shaped cuts 3840 in a side wall of anchor cone implant 3830. The one or more U-shaped cuts extend through the side wall of the anchor cone implant 3830, such that when the screw 3808 is threaded into the anchor cone implant 3830, the one or more U-shaped cut areas of the sidewall will splay away from the sidewall of the anchor cone implant 3830.

The bone anchor 3812 may be implanted by creating a pilot hole into a vertebral body 104. Next, utilizing an instrument to engage instrument engagement features 3832 on the head of the anchor cone implant 3830. The anchor cone implant 3830 may then be threaded into the pilot hole in the vertebral body 104. After which, the engagement features on the tulip head of the screw 3808 may be engaged with an instrument and the screw 3808 may be threaded into a central threaded lumen in the anchor cone implant 3830. Then a cord 116 may be inserted into the cord receiver of the screw 3808. Next, the cord 116 may be tensioned with a cord tensioner. After the cord is sufficiently tensioned, a set screw 204 may be threaded into the internal threads of the cord receiver of the screw 3808.

A bone anchor 3812 that includes an anchor cone implant 3830 may be utilized to improve bone purchase or mitigate screw migration. Alternatively, a bone anchor 3812 that includes an anchor cone implant 3830 may be utilized when a revision surgery is necessary or if the bone quality is poor. It will be understood that the bone anchor 3812 is similar to the bone anchor 3712, with wider, thicker walls on the cone, especially near the proximal end of the cone. As will be readily appreciated, a thicker cone may be utilized in larger vertebral bodies 104 or where there is poorer bone quality or other situations as determined by a surgeon that such a thicker anchor cone implant 3830 is appropriate.

FIG. 38D illustrates a bone anchor with an anchor cone implant 3850 that is shorter and includes a threaded lumen. The lumen of anchor cone implant 3850 extends through the distal end of the cone implant 3850 and is sized to permit the screw to exit the distal end of the cone implant 3850. A shorter anchor cone implant 3850 may be determined by a surgeon to be appropriate in certain situations, such as smaller vertebral bodies 104 or similar situations. The anchor cone implant 3850 may be fabricated of titanium, tantalum or other biocompatible material that is sufficiently capable of being screwed into bone. Anchor cone implant 3850 may include slits at the narrow end or U-shaped slits in the sidewall to cause additional splay when a screw is threaded into the internal threaded lumen of the anchor cone implant 3850. Anchor cone implant 3850 may be implanted in a similar manner as anchor cone implants 3730 and 3830.

It should be understood that anchor cone implants 3730, 3830, or 3850 may be used instead of or in combination with a staple in situations in which anchor cone implants are considered appropriate, such as to improve bone purchase, to lower the likelihood of screw migration, poor bone quality, and similar situations.

FIG. 38E illustrates a side cut away view of the tip of a screw 3812 that has been threaded into an anchor cone implant 3860 with a resulting splayed tip 3862 at the distal, narrow end of the anchor cone implant 3860. It will be readily appreciated that anchor cone implants 3730, 3830, and 3850 that comprise laser cut slits at the distal, narrow tip of the cone would have similar splay, flare or spread at the distal, narrow end of the cone when a screw is threaded therein, as shown in FIG. 38E. Likewise, it will be appreciated that there would be a similar U-shaped splay, flare or spread away from the side walls of the anchor cone implants 3740, 3840 or 3850 having U-shaped laser cuts or slits therein when a screw 3712 or 3812 is threaded into the central threaded lumen.

It will be appreciated that the various configurations of bone anchors 3712 or 3812 as illustrated in FIGS. 37A-38D may be appropriate to improve bone purchase, mitigate implant migration, mitigate implant backout, or similar surgical procedure plan objectives as determined appropriate by a surgeon. Moreover, the bone anchors 3712 or 3812 may be used in combination with other embodiments disclosed in the disclosure.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A bone anchor, comprising:
a screw comprising a head and a shank, wherein the shank defines a screw axis; and
a connector attachable to the head of the screw, wherein the connector is configured to receive and secure a cord offset from the screw axis.

2. The bone anchor of claim 1, wherein the connector comprises a connector body and connector cap that moves relative to the connector body.

3. The bone anchor of claim 2, further comprising a set screw that threads into the connector body to secure the connector cap to the connector body.

4. The bone anchor of claim 3, wherein the connector body comprises a cord receiver and wherein a size of the cord receiver changes as the connector cap moves relative to the connector body.

5. The bone anchor of claim 3, wherein the connector body comprises a cord receiver having an open top end to receive the cord.

6. The bone anchor of claim 5, wherein the connector cap comprises a clamp that retains the cord between the connector body and the connector cap and that limits the cord from sliding through the cord receiver when the connector cap is secured into the connector body.

7. The bone anchor of claim 1, further comprising a staple through which the screw is inserted.

8. The bone anchor of claim 7, wherein the staple comprises an extending surface having at least one additional opening for a second screw.

9. The bone anchor of claim 8, wherein the connector aligns the cord with a screw axis of the second screw.

10. The bone anchor of claim 7, wherein the staple comprises an extension having a cord support face to receive and secure at least one of a first cord and a second cord between a post and the cord support face.

11. The bone anchor of claim 7, wherein the staple comprises an alignment feature.

12. The bone anchor of claim 11, wherein the alignment feature is provided on a ring that is rotatable relative to a bottom portion of the staple.

13. The bone anchor of claim 1, further comprising an anchor plate having a first opening and a second opening.

14. A bone anchor, comprising:
a first screw;
a second screw;
an anchor plate coupling the first screw with the second screw; and
a cord receiver configured to couple a cord or second cord under tension with one or both of the first screw and second screw.

15. The bone anchor of claim 14, wherein the cord receiver is integrally formed with the anchor plate.

16. The bone anchor of claim 14, wherein the cord receiver is part of the first screw.

17. The bone anchor of claim 14, wherein the cord receiver comprises a tulip head having internal threads to receive a set screw.

18. The bone anchor of claim 14, wherein the cord receiver and the anchor plate comprise teeth that substantially prevent a relative rotation thereof when a face of the anchor plate contacts a face of the cord receiver.

19. The bone anchor of claim 14, wherein the first screw extends through a first opening of the anchor plate and wherein the second screw extends through a second opening of the anchor plate.

20. The bone anchor of claim 19, wherein the first screw draws the cord receiver into contact with the anchor plate and wherein the cord receiver abuts the anchor plate to substantially prevent the second screw from withdrawing out of the anchor plate.
